SEQUENCE LISTING (112 sequences)

SEQ ID NO: 1 - DNA, 3633 bp, Homo sapiens
  mutation (2203)..(2203): Mutation G > A
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatga

SEQ ID NO: 2 - protein, 1210 aa, Homo sapiens [sequence omitted]

SEQ ID NO: 3 - protein, 119 aa, Homo sapiens
KRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMP

SEQ ID NO: 4 - protein, 119 aa, Homo sapiens
KRTLRRLLQERELVEPLTPSGEAPNQALLRILKETEFKKIKVLGSGAFGTVYKGLWIPESEKVKIPVAIKELREATSPKANKEILDEAYVMASVDNPHVCRLLGICLTSTVQLITQLMP

SEQ ID NO: 5 - DNA, 119 bp, Homo sapiens
tcaaagtgctgggctccggtgcgttcggcacggtgtataagggactctggatcccagaaggtgagaaagttaaaattcccgtcgctatcaaggaattaagagaagcaacatctccgaaa

SEQ ID NO: 6 - DNA, 119 bp, Homo sapiens
tcaaagtgctgggctccggtgcgttcggcacggtgtataagggactctggatcccagaaagtgagaaagttaaaattcccgtcgctatcaaggaattaagagaagcaacatctccgaaa

SEQ ID NO: 7 - protein, 119 aa, Homo sapiens
PEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHV

SEQ ID NO: 8 - protein, 119 aa, Homo sapiens
PEGEKVKIPVAIKELREATSPKANKEILDEAYVMASVDNPHVCRLLGICLTSTVQLITQFMPFGCLLDYVREHKDNIGSQYLLNWCVQIAKGMNYLEDRRLVHRDLAARNVLVKTPQHV

SEQ ID NO: 9 - DNA, 119 bp, Homo sapiens
cccacgtgtgccgcctgctgggcatctgcctcacctccaccgtgcagctcatcacgcagctcatgcccttcggctgcctcctggactatgtccgggaacacaaagacaatattggctcc

SEQ ID NO: 10 - DNA, 119 bp, Homo sapiens
cccacgtgtgccgcctgctgggcatctgcctcacctccaccgtgcagctcatcacgcagttcatgcccttcggctgcctcctggactatgtccgggaacacaaagacaatattggctcc

SEQ ID NO: 11 - protein, 119 aa, Homo sapiens
GEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKI

SEQ ID NO: 12 - protein, 119 aa, Homo sapiens
GEKVKIPVAIKELREATSPKANKEILDEAYVMASVDNPHVCRLLGICLTSTVQLITQLMSFGCLLDYVREHKDNIGSQYLLNWCVQIAKGMNYLEDRRLVHRDLAARNVLVKTPQHVKI

SEQ ID NO: 13 - DNA, 119 bp, Homo sapiens
tgtgccgcctgctgggcatctgcctcacctccaccgtgcagctcatcacgcagctcatgcccttcggctgcctcctggactatgtccgggaacacaaagacaatattggctcccagtac

SEQ ID NO: 14 - DNA, 119 bp, Homo sapiens
tgtgccgcctgctgggcatctgcctcacctccaccgtgcagctcatcacgcagctcatgtccttcggctgcctcctggactatgtccgggaacacaaagacaatattggctcccagtac

SEQ ID NO: 15 - protein, 119 aa, Homo sapiens
KELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLG

SEQ ID NO: 16 - protein, 119 aa, Homo sapiens
KELREATSPKANKEILDEAYVMASVDNPHVCRLLGICLTSTVQLITQLMPFGCLLDYVRDHKDNIGSQYLLNWCVQIAKGMNYLEDRRLVHRDLAARNVLVKTPQHVKITDFGLAKLLG

SEQ ID NO: 17 - DNA, 119 bp, Homo sapiens
accgtgcagctcatcacgcagctcatgcccttcggctgcctcctggactatgtccgggaacacaaagacaatattggctcccagtacctgctcaactggtgtgtgcagatcgcaaaggg

SEQ ID NO: 18 - DNA, 119 bp, Homo sapiens
accgtgcagctcatcacgcagctcatgcccttcggctgcctcctggactatgtccgggatcacaaagacaatattggctcccagtacctgctcaactggtgtgtgcagatcgcaaaggg

SEQ ID NO: 19 - protein, 119 aa, Homo sapiens
TSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYG

SEQ ID NO: 20 - protein, 119 aa, Homo sapiens
TSTVQLITQLMPFGCLLDYVREHKDNIGSQYLLNWCVQIAKGMNYLEDRRLVHRDLAARIVLVKTPQHVKITDFGLAKLLGAEEKEYHAEGGKVPIKWMALESILHRIYTHQSDVWSYG

SEQ ID NO: 21 - DNA, 119 bp, Homo sapiens
aaagggcatgaactacttggaggaccgtcgcttggtgcaccgcgacctggcagccaggaacgtactggtgaaaacaccgcagcatgtcaagatcacagattttgggctggccaaactgc

SEQ ID NO: 22 - DNA, 119 bp, Homo sapiens
aaagggcatgaactacttggaggaccgtcgcttggtgcaccgcgacctggcagccaggatcgtactggtgaaaacaccgcagcatgtcaagatcacagattttgggctggccaaactgc

SEQ ID NO: 23 - protein, 119 aa, Homo sapiens
STVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGV

SEQ ID NO: 24 - protein, 119 aa, Homo sapiens
STVQLITQLMPFGCLLDYVREHKDNIGSQYLLNWCVQIAKGMNYLEDRRLVHRDLAARNILVKTPQHVKITDFGLAKLLGAEEKEYHAEGGKVPIKWMALESILHRIYTHQSDVWSYGV

SEQ ID NO: 25 - DNA, 119 bp, Homo sapiens
agggcatgaactacttggaggaccgtcgcttggtgcaccgcgacctggcagccaggaacgtactggtgaaaacaccgcagcatgtcaagatcacagattttgggctggccaaactgctg

SEQ ID NO: 26 - DNA, 119 bp, Homo sapiens
agggcatgaactacttggaggaccgtcgcttggtgcaccgcgacctggcagccaggaacatactggtgaaaacaccgcagcatgtcaagatcacagattttgggctggccaaactgctg

SEQ ID NO: 27 - protein, 119 aa, Homo sapiens
LITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWE

SEQ ID NO: 28 - protein, 119 aa, Homo sapiens
LITQLMPFGCLLDYVREHKDNIGSQYLLNWCVQIAKGMNYLEDRRLVHRDLAARNVLVKIPQHVKITDFGLAKLLGAEEKEYHAEGGKVPIKWMALESILHRIYTHQSDVWSYGVTVWE

SEQ ID NO: 29 - DNA, 119 bp, Homo sapiens
cttggaggaccgtcgcttggtgcaccgcgacctggcagccaggaacgtactggtgaaaacaccgcagcatgtcaagatcacagattttgggctggccaaactgctgggtgcggaagaga

SEQ ID NO: 30 - DNA, 119 bp, Homo sapiens
cttggaggaccgtcgcttggtgcaccgcgacctggcagccaggaacgtactggtgaaaataccgcagcatgtcaagatcacagattttgggctggccaaactgctgggtgcggaagaga

SEQ ID NO: 31 - protein, 119 aa, Homo sapiens
LLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYD

SEQ ID NO: 32 - protein, 119 aa, Homo sapiens
LLDYVREHKDNIGSQYLLNWCVQIAKGMNYLEDRRLVHRDLAARNVLVKTPQHVKITDFELAKLLGAEEKEYHAEGGKVPIKWMALESILHRIYTHQSDVWSYGVTVWELMTFGSKPYD

SEQ ID NO: 33 - DNA, 119 bp, Homo sapiens
cctggcagccaggaacgtactggtgaaaacaccgcagcatgtcaagatcacagattttgggctggccaaactgctgggtgcggaagagaaagaataccatgcagaaggaggcaaagtgc

SEQ ID NO: 34 - DNA, 119 bp, Homo sapiens
cctggcagccaggaacgtactggtgaaaacaccgcagcatgtcaagatcacagattttgagctggccaaactgctgggtgcggaagagaaagaataccatgcagaaggaggcaaagtgc

SEQ ID NO: 35 - DNA, 3768 bp, Homo sapiens
  mutation (1960)..(1960): Mutation A > G
atggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtga

SEQ ID NO: 36 - protein, 1255 aa, Homo sapiens [sequence omitted]

SEQ ID NO: 37 - protein, 119 aa, Homo sapiens
FCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMR

SEQ ID NO: 38 - protein, 119 aa, Homo sapiens
FCVARCPSGVKPDLSYMPIWKFPDEEGACQPCPINCTHSCVDLDDKGCPAEQRASPLTSVISAVVGILLVVVLGVVFGILIKRRQQKIRKYTMRRLLQETELVEPLTPSGAMPNQAQMR

SEQ ID NO: 39 - DNA, 119 bp, Homo sapiens
gtgtggacctggatgacaagggctgccccgccgagcagagagccagccctctgacgtccatcatctctgcggtggttggcattctgctggtcgtggtcttgggggtggtctttgggatc

SEQ ID NO: 40 - DNA, 119 bp, Homo sapiens
gtgtggacctggatgacaagggctgccccgccgagcagagagccagccctctgacgtccgtcatctctgcggtggttggcattctgctggtcgtggtcttgggggtggtctttgggatc

SEQ ID NO: 41 - protein, 119 aa, Homo sapiens
VDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIK

SEQ ID NO: 42 - protein, 119 aa, Homo sapiens
VDLDDKGCPAEQRASPLTSIISAVVGILLVVVLGVVFGILIKRRQQKIRKYTMRRLLQEMELVEPLTPSGAMPNQAQMRILKETELRKVKVLGSGAFGTVYKGIWIPDGENVKIPVAIK

SEQ ID NO: 43 - DNA, 119 bp, Homo sapiens
catcaagcgacggcagcagaagatccggaagtacacgatgcggagactgctgcaggaaacggagctggtggagccgctgacacctagcggagcgatgcccaaccaggcgcagatgcgga

SEQ ID NO: 44 - DNA, 119 bp, Homo sapiens
catcaagcgacggcagcagaagatccggaagtacacgatgcggagactgctgcaggaaatggagctggtggagccgctgacacctagcggagcgatgcccaaccaggcgcagatgcgga

SEQ ID NO: 45 - protein, 119 aa, Homo sapiens
LGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRL

SEQ ID NO: 46 - protein, 119 aa, Homo sapiens
LGVVFGILIKRRQQKIRKYTMRRLLQETELVEPLTPSGAMPNQAQMRILKETELRKVKVFGSGAFGTVYKGIWIPDGENVKIPVAIKVLRENTSPKANKEILDEAYVMAGVGSPYVSRL

SEQ ID NO: 47 - DNA, 119 bp, Homo sapiens
tgcccaaccaggcgcagatgcggatcctgaaagagacggagctgaggaaggtgaaggtgcttggatctggcgcttttggcacagtctacaagggcatctggatccctgatggggagaat

SEQ ID NO: 48 - DNA, 119 bp, Homo sapiens
tgcccaaccaggcgcagatgcggatcctgaaagagacggagctgaggaaggtgaaggtgtttggatctggcgcttttggcacagtctacaagggcatctggatccctgatggggagaat

SEQ ID NO: 49 - protein, 119 aa, Homo sapiens
YKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLV

SEQ ID NO: 50 - protein, 119 aa, Homo sapiens
YKGIWIPDGENVKIPVAIKVLRENTSPKANKEILDEAYVMAGVGSPYVSRLLGICLTSTMQLVTQLMPYGCLLDHVRENRGRLGSQDLLNWCMQIAKGMSYLEDVRLVHRDLAARNVLV

SEQ ID NO: 51 - DNA, 119 bp, Homo sapiens
tggctggtgtgggctccccatatgtctcccgccttctgggcatctgcctgacatccacggtgcagctggtgacacagcttatgccctatggctgcctcttagaccatgtccgggaaaac

SEQ ID NO: 52 - DNA, 119 bp, Homo sapiens
tggctggtgtgggctccccatatgtctcccgccttctgggcatctgcctgacatccacgatgcagctggtgacacagcttatgccctatggctgcctcttagaccatgtccgggaaaac

SEQ ID NO: 53 - protein, 119 aa, Homo sapiens
PVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLA

SEQ ID NO: 54 - protein, 119 aa, Homo sapiens
PVAIKVLRENTSPKANKEILDEAYVMAGVGSPYVSRLLGICLTSTVQLVTQLMPYGCLLNHVRENRGRLGSQDLLNWCMQIAKGMSYLEDVRLVHRDLAARNVLVKSPNHVKITDFGLA

SEQ ID NO: 55 - DNA, 119 bp, Homo sapiens
tctgcctgacatccacggtgcagctggtgacacagcttatgccctatggctgcctcttagaccatgtccgggaaaaccgcggacgcctgggctcccaggacctgctgaactggtgtatg

SEQ ID NO: 56 - DNA, 119 bp, Homo sapiens
tctgcctgacatccacggtgcagctggtgacacagcttatgccctatggctgcctcttaaaccatgtccgggaaaaccgcggacgcctgggctcccaggacctgctgaactggtgtatg

SEQ ID NO: 57 - DNA, 3927 bp, Homo sapiens
  mutation (2353)..(2353): Mutation G > A
atgaagccggcgacaggactttgggtctgggtgagccttctcgtggcggcggggaccgtccagcccagcgattctcagtcagtgtgtgcaggaacggagaataaactgagctctctctctgacctggaacagcagtaccgagccttgcgcaagtactatgaaaactgtgaggttgtcatgggcaacctggagataaccagcattgagcacaaccgggacctctccttcctgcggtctgttcgagaagtcacaggctacgtgttagtggctcttaatcagtttcgttacctgcctctggagaatttacgcattattcgtgggacaaaactttatgaggatcgatatgccttggcaatatttttaaactacagaaaagatggaaactttggacttcaagaacttggattaaagaacttgacagaaatcctaaatggtggagtctatgtagaccagaacaaattcctttgttatgcagacaccattcattggcaagatattgttcggaacccatggccttccaacttgactcttgtgtcaacaaatggtagttcaggatgtggacgttgccataagtcctgtactggccgttgctggggacccacagaaaatcattgccagactttgacaaggacggtgtgtgcagaacaatgtgacggcagatgctacggaccttacgtcagtgactgctgccatcgagaatgtgctggaggctgctcaggacctaaggacacagactgctttgcctgcatgaatttcaatgacagtggagcatgtgttactcagtgtccccaaacctttgtctacaatccaaccacctttcaactggagcacaatttcaatgcaaagtacacatatggagcattctgtgtcaagaaatgtccacataactttgtggtagattccagttcttgtgtgcgtgcctgccctagttccaagatggaagtagaagaaaatgggattaaaatgtgtaaaccttgcactgacatttgcccaaaagcttgtgatggcattggcacaggatcattgatgtcagctcagactgtggattccagtaacattgacaaattcataaactgtaccaagatcaatgggaatttgatctttctagtcactggtattcatggggacccttacaatgcaattgaagccatagacccagagaaactgaacgtctttcggacagtcagagagataacaggtttcctgaacatacagtcatggccaccaaacatgactgacttcagtgttttttctaacctggtgaccattggtggaagagtactctatagtggcctgtccttgcttatcctcaagcaacagggcatcacctctctacagttccagtccctgaaggaaatcagcgcaggaaacatctatattactgacaacagcaacctgtgttattatcataccattaactggacaacactcttcagcacaatcaaccagagaatagtaatccgggacaacagaaaagctgaaaattgtactgctgaaggaatggtgtgcaaccatctgtgttccagtgatggctgttggggacctgggccagaccaatgtctgtcgtgtcgccgcttcagtagaggaaggatctgcatagagtcttgtaacctctatgatggtgaatttcgggagtttgagaatggctccatctgtgtggagtgtgacccccagtgtgagaagatggaagatggcctcctcacatgccatggaccgggtcctgacaactgtacaaagtgctctcattttaaagatggcccaaactgtgtggaaaaatgtccagatggcttacagggggcaaacagtttcattttcaagtatgctgatccagatcgggagtgccacccatgccatccaaactgcacccaagggtgtaacggtcccactagtcatgactgcatttactacccatggacgggccattccactttaccacaacatgctagaactcccctgattgcagctggagtaattggtgggctcttcattctggtcattgtgggtctgacatttgctgtttatgttagaaggaagagcatcaaaaagaaaagagccttgagaagattcttggaaacagagttggtggaaccattaactcccagtggcacagcacccaatcaagctcaacttcgtattttgaaagaaactgagctgaagagggtaaaagtccttggctcaggtgcttttggaacggtttataaaggtatttgggtacctgaaggagaaactgtgaagattcctgtggctattaagattcttaatgagacaactggtcccaaggcaaatgtggagttcatggatgaagctctgatcatggcaagtatggatcatccacacctagtccggttgctgggtgtgtgtctgagcccaaccatccagctggttactcaacttatgccccatggctgcctgttggagtatgtccacgagcacaaggataacattggatcacaactgctgcttaactggtgtgtccagatagctaagggaatgatgtacctggaagaaagacgactcgttcatcgggatttggcagcccgtaatgtcttagtgaaatctccaaaccatgtgaaaatcacagattttgggctagccagactcttggaaggagatgaaaaagagtacaatgctgatggaggaaagatgccaattaaatggatggctctggagtgtatacattacaggaaattcacccatcagagtgacgtttggagctatggagttactatatgggaactgatgacctttggaggaaaaccctatgatggaattccaacgcgagaaatccctgatttattagagaaaggagaacgtttgcctcagcctcccatctgcactattgacgtttacatggtcatggtcaaatgttggatgattgatgctgacagtagacctaaatttaaggaactggctgctgagttttcaaggatggctcgagaccctcaaagatacctagttattcagggtgatgatcgtatgaagcttcccagtccaaatgacagcaagttctttcagaatctcttggatgaagaggatttggaagatatgatggatgctgaggagtacttggtccctcaggctttcaacatcccacctcccatctatacttccagagcaagaattgactcgaataggagtgaaattggacacagccctcctcctgcctacacccccatgtcaggaaaccagtttgtataccgagatggaggttttgctgctgaacaaggagtgtctgtgccctacagagccccaactagcacaattccagaagctcctgtggcacagggtgctactgctgagatttttgatgactcctgctgtaatggcaccctacgcaagccagtggcaccccatgtccaagaggacagtagcacccagaggtacagtgctgaccccaccgtgtttgccccagaacggagcccacgaggagagctggatgaggaaggttacatgactcctatgcgagacaaacccaaacaagaatacctgaatccagtggaggagaacccttttgtttctcggagaaaaaatggagaccttcaagcattggataatcccgaatatcacaatgcatccaatggtccacccaaggccgaggatgagtatgtgaatgagccactgtacctcaacacctttgccaacaccttgggaaaagctgagtacctgaagaacaacatactgtcaatgccagagaaggccaagaaagcgtttgacaaccctgactactggaaccacagcctgccacctcggagcacccttcagcacccagactacctgcaggagtacagcacaaaatatttttataaacagaatgggcggatccggcctattgtggcagagaatcctgaatacctctctgagttctccctgaagccaggcactgtgctgccgcctccaccttacagacaccggaatactgtggtgtaa

SEQ ID NO: 58 - protein, 1308 aa, Homo sapiens
  VARIANT (785)..(785): Mutation G > S
MKPATGLWVWVSLLVAAGTVQPSDSQSVCAGTENKLSSLSDLEQQYRALRKYYENCEVVMGNLEITSIEHNRDLSFLRSVREVTGYVLVALNQFRYLPLENLRIIRGTKLYEDRYALAIFLNYRKDGNFGLQELGLKNLTEILNGGVYVDQNKFLCYADTIHWQDIVRNPWPSNLTLVSTNGSSGCGRCHKSCTGRCWGPTENHCQTLTRTVCAEQCDGRCYGPYVSDCCHRECAGGCSGPKDTDCFACMNFNDSGACVTQCPQTFVYNPTTFQLEHNFNAKYTYGAFCVKKCPHNFVVDSSSCVRACPSSKMEVEENGIKMCKPCTDICPKACDGIGTGSLMSAQTVDSSNIDKFINCTKINGNLIFLVTGIHGDPYNAIEAIDPEKLNVFRTVREITGFLNIQSWPPNMTDFSVFSNLVTIGGRVLYSGLSLLILKQQGITSLQFQSLKEISAGNIYITDNSNLCYYHTINWTTLFSTINQRIVIRDNRKAENCTAEGMVCNHLCSSDGCWGPGPDQCLSCRRFSRGRICIESCNLYDGEFREFENGSICVECDPQCEKMEDGLLTCHGPGPDNCTKCSHFKDGPNCVEKCPDGLQGANSFIFKYADPDRECHPCHPNCTQGCNGPTSHDCIYYPWTGHSTLPQHARTPLIAAGVIGGLFILVIVGLTFAVYVRRKSIKKKRALRRFLETELVEPLTPSGTAPNQAQLRILKETELKRVKVLGSGAFGTVYKGIWVPEGETVKIPVAIKILNETTGPKANVEFMDEALIMASMDHPHLVRLLGVCLSPTIQLVTQLMPHGCLLEYVHEHKDNIGSQLLLNWCVQIAKGMMYLEERRLVHRDLAARNVLVKSPNHVKITDFGLARLLEGDEKEYNADGGKMPIKWMALECIHYRKFTHQSDVWSYGVTIWELMTFGGKPYDGIPTREIPDLLEKGERLPQPPICTIDVYMVMVKCWMIDADSRPKFKELAAEFSRMARDPQRYLVIQGDDRMKLPSPNDSKFFQNLLDEEDLEDMMDAEEYLVPQAFNIPPPIYTSRARIDSNRSEIGHSPPPAYTPMSGNQFVYRDGGFAAEQGVSVPYRAPTSTIPEAPVAQGATAEIFDDSCCNGTLRKPVAPHVQEDSSTQRYSADPTVFAPERSPRGELDEEGYMTPMRDKPKQEYLNPVEENPFVSRRKNGDLQALDNPEYHNASNGPPKAEDEYVNEPLYLNTFANTLGKAEYLKNNILSMPEKAKKAFDNPDYWNHSLPPRSTLQHPDYLQEYSTKYFYKQNGRIRPIVAENPEYLSEFSLKPGTVLPPPPYRHRNTVV

SEQ ID NO: 59 - protein, 119 aa, Homo sapiens
SGAFGTVYKGIWVPEGETVKIPVAIKILNETTGPKANVEFMDEALIMASMDHPHLVRLLGVCLSPTIQLVTQLMPHGCLLEYVHEHKDNIGSQLLLNWCVQIAKGMMYLEERRLVHRDL

SEQ ID NO: 60 - protein, 119 aa, Homo sapiens
SGAFGTVYKGIWVPEGETVKIPVAIKILNETTGPKANVEFMDEALIMASMDHPHLVRLLSVCLSPTIQLVTQLMPHGCLLEYVHEHKDNIGSQLLLNWCVQIAKGMMYLEERRLVHRDL

SEQ ID NO: 61 - DNA, 119 bp, Homo sapiens
tcatggatgaagctctgatcatggcaagtatggatcatccacacctagtccggttgctgggtgtgtgtctgagcccaaccatccagctggttactcaacttatgccccatggctgcctg

SEQ ID NO: 62 - DNA, 119 bp, Homo sapiens
tcatggatgaagctctgatcatggcaagtatggatcatccacacctagtccggttgctgagtgtgtgtctgagcccaaccatccagctggttactcaacttatgccccatggctgcctg

SEQ ID NO: 63 - protein, 119 aa, Homo sapiens
HLVRLLGVCLSPTIQLVTQLMPHGCLLEYVHEHKDNIGSQLLLNWCVQIAKGMMYLEERRLVHRDLAARNVLVKSPNHVKITDFGLARLLEGDEKEYNADGGKMPIKWMALECIHYRKF

SEQ ID NO: 64 - protein, 119 aa, Homo sapiens
HLVRLLGVCLSPTIQLVTQLMPHGCLLEYVHEHKDNIGSQLLLNWCVQIAKGMMYLEERQLVHRDLAARNVLVKSPNHVKITDFGLARLLEGDEKEYNADGGKMPIKWMALECIHYRKF

SEQ ID NO: 65 - DNA, 119 bp, Homo sapiens
actgctgcttaactggtgtgtccagatagctaagggaatgatgtacctggaagaaagacgactcgttcatcgggatttggcagcccgtaatgtcttagtgaaatctccaaaccatgtga

SEQ ID NO: 66 - DNA, 119 bp, Homo sapiens
actgctgcttaactggtgtgtccagatagctaagggaatgatgtacctggaagaaagacaactcgttcatcgggatttggcagcccgtaatgtcttagtgaaatctccaaaccatgtga

SEQ ID NO: 67 - protein, 119 aa, Homo sapiens
AKGMMYLEERRLVHRDLAARNVLVKSPNHVKITDFGLARLLEGDEKEYNADGGKMPIKWMALECIHYRKFTHQSDVWSYGVTIWELMTFGGKPYDGIPTREIPDLLEKGERLPQPPICT

SEQ ID NO: 68 - protein, 119 aa, Homo sapiens
AKGMMYLEERRLVHRDLAARNVLVKSPNHVKITDFGLARLLEGDEKEYNADGGKMPIKWIALECIHYRKFTHQSDVWSYGVTIWELMTFGGKPYDGIPTREIPDLLEKGERLPQPPICT

SEQ ID NO: 69 - DNA, 119 bp, Homo sapiens
ttggaaggagatgaaaaagagtacaatgctgatggaggaaagatgccaattaaatggatggctctggagtgtatacattacaggaaattcacccatcagagtgacgtttggagctatgg

SEQ ID NO: 70 - DNA, 119 bp, Homo sapiens
ttggaaggagatgaaaaagagtacaatgctgatggaggaaagatgccaattaaatggatagctctggagtgtatacattacaggaaattcacccatcagagtgacgtttggagctatgg

SEQ ID NO: 71 - DNA, 39 bp, Homo sapiens
gttttcccagtcacgacggtagagaaggcgtacatttgt

SEQ ID NO: 72 - DNA, 40 bp, Homo sapiens
aggaaacagctatgaccattgatggaaatatacagcttgc

SEQ ID NO: 73 - DNA, 39 bp, Homo sapiens
gttttcccagtcacgacggtaacatccacccagatcact

SEQ ID NO: 74 - DNA, 39 bp, Homo sapiens
aggaaacagctatgaccattaggatgtggagatgagcag

SEQ ID NO: 75 - DNA, 38 bp, Homo sapiens
gttttcccagtcacgacgtcatgcgtcttcacctggaa

SEQ ID NO: 76 - DNA, 39 bp, Homo sapiens
aggaaacagctatgaccattgaggatcctggctccttat

SEQ ID NO: 77 - DNA, 39 bp, Homo sapiens
gttttcccagtcacgacgagagcttcttcccatgatgat

SEQ ID NO: 78 - DNA, 39 bp, Homo sapiens
aggaaacagctatgaccatatacagctagtgggaaggca

SEQ ID NO: 79 - DNA, 39 bp, Homo sapiens
gttttcccagtcacgacgtcgtaattaggtccagagtga

SEQ ID NO: 80 - DNA, 41 bp, Homo sapiens
aggaaacagctatgaccatgcatgtcagaggatataatgta

SEQ ID NO: 81 - DNA, 39 bp, Homo sapiens
gttttcccagtcacgacgagcaagggattgtgattgttc

SEQ ID NO: 82 - DNA, 39 bp, Homo sapiens
aggaaacagctatgaccatagctgtttggctaagagcag

SEQ ID NO: 83 - DNA, 40 bp, Homo sapiens
gttttcccagtcacgacgcttctttaagcaatgccatctt

SEQ ID NO: 84 - DNA, 40 bp, Homo sapiens
aggaaacagctatgaccatcatgtgacagaacacagtgac

SEQ ID NO: 85 - DNA, 38 bp, Homo sapiens
gttttcccagtcacgacgtccgacttccctttccgaat

SEQ ID NO: 86 - DNA, 39 bp, Homo sapiens
aggaaacagctatgaccattctttcaggatccgcatctg

SEQ ID NO: 87 - DNA, 38 bp, Homo sapiens
gttttcccagtcacgacgaagtacacgatgcggagact

SEQ ID NO: 88 - DNA, 39 bp, Homo sapiens
aggaaacagctatgaccataaacactgcctccagctctt

SEQ ID NO: 89 - DNA, 40 bp, Homo sapiens
gttttcccagtcacgacgacaagtaatgatctcctggaag

SEQ ID NO: 90 - DNA, 39 bp, Homo sapiens
aggaaacagctatgaccataatgaagagagaccagagcc

SEQ ID NO: 91 - DNA, 39 bp, Homo sapiens
gttttcccagtcacgacgatggctgtggtttgtgatggt

SEQ ID NO: 92 - DNA, 39 bp, Homo sapiens
aggaaacagctatgaccatagcacccatgtagaccttct

SEQ ID NO: 93 - DNA, 37 bp, Homo sapiens
gttttcccagtcacgacgtatgcacctgggctctttg

SEQ ID NO: 94 - DNA, 39 bp, Homo sapiens
aggaaacagctatgaccatgtcctccaactgtgtgttgt

SEQ ID NO: 95 - DNA, 38 bp, Homo sapiens
gttttcccagtcacgacggacagagtaccatgcagatg

SEQ ID NO: 96 - DNA, 39 bp, Homo sapiens
aggaaacagctatgaccataatcctgggaagtgcacaga

SEQ ID NO: 97 - DNA, 38 bp, Homo sapiens
gttttcccagtcacgacgcatgatgctagactcctgag

SEQ ID NO: 98 - DNA, 39 bp, Homo sapiens
aggaaacagctatgaccatgtctacatacatcctggtcc

SEQ ID NO: 99 - DNA, 38 bp, Homo sapiens
gttttcccagtcacgacgggcaaaccaagttggtgtgt

SEQ ID NO: 100 - DNA, 41 bp, Homo sapiens
aggaaacagctatgaccatggttgtctaaagtaataactcc

SEQ ID NO: 101 - DNA, 40 bp, Homo sapiens
gttttcccagtcacgacgtgtaacatgtaacaggtgctaa

SEQ ID NO: 102 - DNA, 41 bp, Homo sapiens
aggaaacagctatgaccatatttgtaagttgtggagtttgg

SEQ ID NO: 103 - DNA, 40 bp, Homo sapiens
gttttcccagtcacgacgccattagtacaatccaagtaac

SEQ ID NO: 104 - DNA, 41 bp, Homo sapiens
aggaaacagctatgaccataactgttccaggttaggaaata

SEQ ID NO: 105 - DNA, 40 bp, Homo sapiens
gttttcccagtcacgacgccaactgaaggctaagaaactt

SEQ ID NO: 106 - DNA, 41 bp, Homo sapiens
aggaaacagctatgaccatcaggcttattggtttcttgtat

SEQ ID NO: 107 - DNA, 39 bp, Homo sapiens
gttttcccagtcacgacgcagcccaaagactcacattta

SEQ ID NO: 108 - DNA, 41 bp, Homo sapiens
aggaaacagctatgaccatggaaattaggcttatcaatagg

SEQ ID NO: 109 - DNA, 40 bp, Homo sapiens
gttttcccagtcacgacgtagtgctggtttgttcaacata

SEQ ID NO: 110 - DNA, 41 bp, Homo sapiens
aggaaacagctatgaccatcagattgagtaatctctgctat

SEQ ID NO: 111 - DNA, 40 bp, Homo sapiens
gttttcccagtcacgacgctttctttctcagatcattacg

SEQ ID NO: 112 - DNA, 41 bp, Homo sapiens
aggaaacagctatgaccataacatgtttgtggtcctttcca

(19) United States
(12) Patent Application Publication
(10) Pub. No.: US 2013/0005592 A1
(43) Pub. Date: Jan. 3, 2013

(54) METHODS AND COMPOSITIONS FOR PREDICTING CANCER THERAPY RESPONSE

(75) Inventors: Steven Stone, Salt Lake City, UT (US); Alexander Gutin, Salt Lake City, UT (US); Darl Flake, Salt Lake City, UT (US); Francisco J. Esteva, Houston, TX (US); Dihua Yu, Houston, TX (US)

(73) Assignees: Board of Regents of the University of Texas System, Austin, TX (US); Myriad Genetics Incorporated, Salt Lake City, UT (US)

(21) Appl. No.: 13/394,936

(22) PCT Filed: Sep. 10, 2010
(86) PCT No.: PCT/US10/48445

§ 371 Date: Jun. 20, 2012

Related U.S. Application Data
(60) Provisional application No. 61/241,293, filed on Sep. 10, 2009.

(51) Int. Cl. C40B 30/04; C12Q 1/68; G01N 33/574; C07H 21/04

(52) U.S. Cl. 506/9; 536/24.31; 435/6.11; 435/7.92; 435/7.1; 435/7.23

(57) ABSTRACT

The invention generally relates to molecular diagnostics, and particularly to molecular markers for cancer therapy response and methods of use thereof.

### CROSS REFERENCE TO RELATED APPLICATIONS

This application claims the priority benefit of U.S. Provisional Application Ser. No. 61/241,293 (filed on Sep. 10, 2009), which is hereby incorporated by reference in its entirety.

### FIELD OF THE INVENTION

The present invention relates generally to the fields of molecular biology and oncology. More particularly, it concerns diagnostic, prognostic, and therapeutic methods and compositions involving HER2-overexpressing cancers and potential efficacy of HER2-targeting agents to treat such cancers.

### SEQUENCE LISTING

A formal Sequence Listing in computer readable form has been submitted electronically with this application as a text file. This text file, which is named “3315-01-1WO-2010-09-10-SEQ-LIST-TXT-BGJ_ST25.txt”, was created on Sep. 10, 2010, and is 98,536 bytes in size. Its contents are incorporated by reference herein in their entirety.

### BACKGROUND OF THE INVENTION

Overexpression of HER2 (Entrez GeneID no. 2064; also called ErbB2) is found in approximately 20-30% of human breast cancers and many other cancer types. See, e.g., Slamon et al., SCIENCE (1987) 235:177-182; Yu & Hung, ONCOGENE (2000) 19:6115-6121. HER2 overexpression leads to an aggressive cancer phenotype and poor patient survival. See, e.g., Yu & Hung, ONCOGENE (2000) 19:6115-6121. Thus HER2-targeting cancer therapy is an area of intense research. One successful example is trastuzumab (Herceptin™), a recombinant humanized anti-HER2 monoclonal antibody that binds the extracellular domain of HER2. See, e.g., Shepard et al., J. CLIN. IMMUNOL. (1991) 11:117-127).

Trastuzumab shows remarkable efficacy both as a single agent and in combination therapy. See, e.g., Cobleigh et al., J. CLIN. ONCOL. (1999) 17:2639-2648; Seidman et al., J. CLIN. ONCOL. (2001) 19:2587-2595; Slamon et al., N. ENGL. J. MED. (2001) 344:783-792; Esteva et al., J. CLIN. ONCOL. (2002) 20:1800-1808. However, only about 35% of HER2-overexpressing patients respond well to trastuzumab while roughly 5% of patients show severe side effects including heart problems. See, e.g., Cobleigh et al., J. CLIN. ONCOL. (1999) 17:2639-2648; Vogel et al., J. CLIN. ONCOL. (2002) 20:719-726. Thus, given both the promise potential drawbacks of anti-HER2 therapy, there is a great need to predict which patients will respond well to treatment.

### BRIEF SUMMARY OF THE INVENTION

The present invention is based in part on the discovery that an activated status for any one of EGFR, HER2 or HER4, a deficiency in PTEN activity, or any combination of these in a patient's cancer cells is significantly associated with such a patient's likelihood of resistance or response to some HER2-targeting agents (e.g., trastuzumab). Thus, the present invention concerns diagnostic, prognostic, and therapeutic methods and compositions for cancers that involve HER2 amplification/overexpression, and consequently, HER2-targeting agents.

One aspect of the invention provides a method comprising evaluating the status of EGFR, HER2 and HER4 in a patient sample (e.g., to determine whether the patient has an activating mutation in any one of these genes). In some embodiments the method further comprises evaluating the status of PTEN in a sample from the patient. In some embodiments the method further comprises determining whether HER2 is amplified/overexpressed in a sample from the patient.

The status of a gene can, according to the invention, be evaluated by various techniques. In some embodiments status is evaluated by determining whether one or more of the genes (EGFR, HER2, HER4, PTEN) has a mutation. Mutations may be detected by any suitable technique (e.g., genomic or transcript sequencing, allele-specific amplification, etc.). In other embodiments status is evaluated by determining the expression level of a product of one or more of the genes (e.g., mRNA, protein). Expression levels may be determined by any suitable technique (e.g., quantitative polymerase chain reaction (qPCR), immunohistochemistry (IHC), etc.). In other embodiments status is evaluated using copy number, methylation, gene regulation (e.g., miRNA), etc.

Another aspect of the invention provides a method of determining whether a patient will respond to anti-HER2 receptor therapy comprising evaluating EGFR, HER2 and HER4 status in a sample from the patient, wherein an activated status for any of EGFR, HER2 or HER4 indicates the patient has a reduced or low likelihood of responding to the anti-HER2 receptor therapy. In some embodiments the method further comprises evaluating the status of PTEN in a sample from the patient, wherein activated status for any of EGFR, HER2 or HER4 or low or negative status for PTEN indicates the patient has a reduced or low likelihood of responding to the anti-HER2 receptor therapy. In some embodiments the method further comprises evaluating HER2 amplification/overexpression, wherein any of no HER2 amplification/overexpression, activated status for any of EGFR, HER2 or HER4, or low or negative status for PTEN indicates the patient has a reduced or low likelihood of responding to the anti-HER2 receptor therapy.

Activated status means increased activity by the encoded protein or anything that leads to such increased activity. Thus activated status can mean mutations that lead to increased or constitutive activity in the encoded protein, mutations leading to increased expression of the encoded protein, increased genomic copy number, increased mRNA expression, increased protein expression, etc. Low or negative status means decreased (including absent) activity by the encoded protein or anything that leads to such decreased activity. Thus low or negative status can mean mutations that lead to decreased or abolished activity in the encoded protein, mutations leading to decreased or abolished expression of the encoded protein, decreased genomic copy number, decreased mRNA expression, decreased protein expression, etc.

In some embodiments the invention provides a method of determining whether a patient will respond to anti-HER2 receptor therapy comprising evaluating EGFR, HER2 and HER4 status in a sample from the patient, wherein an activating mutation in any of EGFR, HER2 or HER4 indicates the patient has a reduced or low likelihood of responding to the anti-HER2 receptor therapy. In some embodiments the method further comprises evaluating PTEN protein expression, wherein an activating mutation in any of EGFR, HER2 or HER4 or low or absent PTEN protein expression indicates the patient has a reduced or low likelihood of responding to the anti-HER2 receptor therapy.

In some embodiments, the anti-HER2 receptor therapy comprises trastuzumab (Herceptin™). In other embodiments the anti-HER2 receptor therapy comprises pertuzumab (Omnitarg™).

One aspect of the invention provides a method of determining whether a patient will respond to kinase inhibitor (KI) therapy comprising evaluating HER4 status in a sample from the patient, wherein an activating mutation in HER4 indicates the patient has a high or increased likelihood of responding to the KI therapy. In some embodiments the invention provides a method of determining whether a patient will respond to KI therapy comprising evaluating EGFR, HER2 and HER4 status in a sample from the patient, wherein an activating mutation in any of EGFR, HER2 or HER4 indicates the patient has a high or increased likelihood of responding to the KI therapy. In some embodiments the invention provides a method of determining whether a patient will respond to KI therapy comprising evaluating EGFR, HER2, HER4, and PTEN status in a sample from the patient, wherein an activating mutation in any of EGFR, HER2 or HER4 and normal status for PTEN indicates the patient has a high or increased likelihood of responding to the KI therapy.

Yet another aspect of the invention provides a method of optimizing treatment of a cancer patient comprising evaluating EGFR, HER2 and HER4 status in a sample from the patient and recommending, prescribing or administering a treatment regimen that does not include an anti-HER2 receptor agent if the sample shows an activated status for any of EGFR, HER2 or HER4. In some embodiments the treatment optimization method comprises evaluating EGFR, HER2, HER4, and PTEN status in a sample from the patient and recommending, prescribing or administering a treatment regimen that does not include an anti-HER2 receptor agent if the sample shows an activated status for any of EGFR, HER2 or HER4 or a low or negative status for PTEN. In some embodiments the treatment optimization method comprises evaluating HER2 overexpression and EGFR, HER2 and HER4 status in a sample from the patient and recommending, prescribing or administering a treatment regimen that includes an anti-HER2 receptor agent if the sample shows HER2 overexpression and does not show an activated status for each of EGFR, HER2 and HER4. In some embodiments the treatment optimization method comprises evaluating HER2 overexpression and EGFR, HER2, HER4, and PTEN status in a sample from the patient and recommending, prescribing or administering a treatment regimen that includes an anti-HER2 receptor agent if the sample shows HER2 overexpression, does not show an activated status for each of EGFR, HER2 and HER4, and does not show a low or negative status for PTEN.

In some embodiments the treatment optimization method is implemented on a computer. Thus the invention provides a computer-implemented method of optimizing treatment of a cancer patient comprising: accessing status information for EGFR, HER2 and HER4 derived from a patient sample and stored in a computer-readable medium; querying this information to determine whether the patient has an activated status for any of these genes; outputting [or displaying] the likelihood of the patient responding to anti-HER2 receptor therapy based on the status of these genes. In some embodiments the method may end by additionally or alternatively giving some recommendation as to whether the patient should receive anti-HER2 receptor therapy (e.g., recommending no anti-HER2 receptor therapy if any one of EGFR, HER2 or HER4 is activated). In some embodiments an algorithm is used to calculate the likelihood of the patient responding to anti-HER2 receptor therapy based the status of EGFR, HER2, HER4, and optionally PTEN (along with any additional markers).

Still another aspect of the invention provides apparatus and systems for determining whether a patient will respond to anti-HER2 receptor or KI therapy. These systems will, in some embodiments, use the computer-implemented methods of the invention. In one embodiment the invention provides a system for determining whether a patient will respond to anti-HER2 receptor or KI therapy, comprising: (1) a sample analyzer for determining the status of EGFR, HER2, HER4, and optionally PTEN, wherein the sample analyzer contains the sample or biomolecules from the sample (e.g., DNA, RNA, protein); (2) a first computer program means for (a) receiving status data on EGFR, HER2, HER4, and optionally PTEN, (b) combining the determined status of each of EGFR, HER2, HER4, and optionally PTEN, to provide a test value; and optionally (3) a second computer program means for comparing the test value to one or more reference values each associated with a predetermined degree of probability of response to anti-HER2 receptor or KI therapy.

Another aspect of the invention provides compositions and kits comprising EGFR, HER2, HER4, or PTEN nucleic acids or proteins or nucleic acids or proteins targeted thereto. Such compositions will often include nucleic acids and polypeptides comprising mutants in the EGFR, HER2 or HER4 gene or protein identified in this study. For example, the invention provides a probe set comprising 2 or more nucleic acid probes targeted to each of EGFR, HER2 and HER4 (and optionally PTEN). The invention also provides a microarray comprising such a probe set. The invention also provides kits comprising reagents suitable for detecting, measuring, sequencing, or otherwise analyzing EGFR, HER2, HER4, and optionally PTEN.

### BRIEF DESCRIPTION OF THE DRAWINGS

The following drawings form part of the present specification and are included to further demonstrate certain aspects of the present invention.

The invention may be better understood by reference to one or more of these drawings in combination with the detailed description presented herein.

FIG. 1 is an illustration of an example of a system useful in certain aspects and embodiments of the invention.

FIG. 2 is a flowchart illustrating an example of a computer-implemented method of the invention.

FIG. 3 is a flowchart illustrating an example of a computer-implemented method of the invention.

FIG. 4 is a flowchart illustrating an example of a computer-implemented method of the invention.

FIG. 5 is a flowchart illustrating an example of a computer-implemented method of the invention.

FIG. 6 shows the variants of the invention in the context of the EGFR, HER2 and HER4 genes.

### DETAILED DESCRIPTION OF THE INVENTION

The present invention is based in part on the discovery that an activated status for any one of EGFR (Entrez GeneID no. 1956), HER2 (Entrez GeneID no. 2064) or HER4 (Entrez GeneID no. 2066), optionally along with a deficiency in PTEN (Entrez GeneID no. 5728) activity, in a patient's cancer cells is significantly associated with such a patient's likelihood of resistance or response to HER2-targeting agents (e.g., trastuzumab). More particularly, it has been discovered that an activating mutation in any of EGFR, HER2 or HER4 in a breast cancer patient's tumor cells is significantly correlated with such a patient having weak or no response to trastuzumab. Examples of activating mutations in EGFR, HER2 and HER4 found to be useful according to the invention are listed in Table 1 below.

TABLE 1*

| | RefSeq | | SEQ | Alternative Transcript RefSeq |
|---|---|---|---|---|
| Gene | Accession No. | Variant | ID NOs | Accession Nos. |
| EGFR | NM_005228.3, | G735S | 3-4 | NM_201282.1→NP_ 958439.1; |
| | NP_005219.2 | c.2203G>A | 5-6 | NM_201283.1→NP_ 958440.1; |
| | | L792F | 7-8 | NM_201284.1→NP_958441.1 |
| | | c.2374C>T | 9-10 | |
| | | P794S | 11-12 | |
| | | c.2380C>T | 13-14 | |
| | | E804D | 15-16 | |
| | | c.2412A>T | 17-18 | |
| | | N842I | 19-20 | |
| | | c.2525A>T | 21-22 | |
| | | V843I | 23-24 | |
| | | c.2527G>A | 25-26 | |
| | | T847I | 27-28 | |
| | | c.2540C>T | 29-30 | |
| | | G857E | 31-32 | |
| | | c.2570G>A | 33-34 | |
| HER2 | NM_004448.2, | I654V | 37-38 | NM_001005862.1→NP_ 001005862.1 |
| | NP_004439.2 | c.1960A>G | 39-40 | |
| | | T694M | 41-42 | |
| | | c.2081C>T | 43-44 | |
| | | L726F | 45-46 | |
| | | c.2176C>T | 47-48 | |
| | | V794M | 49-50 | |
| | | c.2380G>A | 51-52 | |
| | | D808N | 53-54 | |
| | | c.2422G>A | 55-56 | |
| HER4 | NM_005235.2, | G785S | 59-60 | NM_001042599.1→NP_001036064.1 |
| | NP_005226.1 | c.2353G>A | 61-62 | |
| | | R838Q | 63-64 | |
| | | c.2513G>A | 65-66 | |
| | | M887I | 67-68 | |
| | | c.2661G>A | 69-70 | |

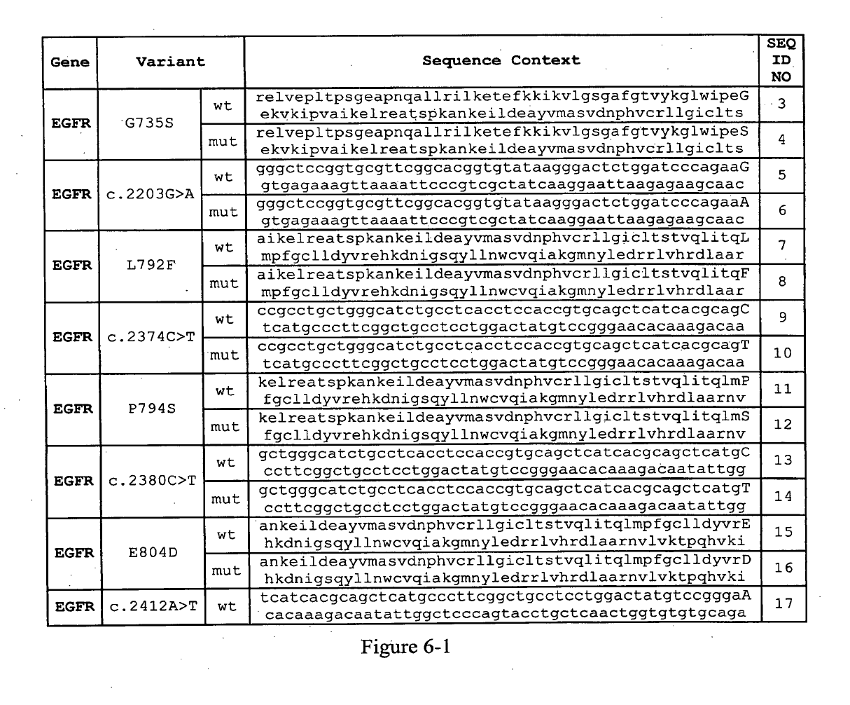

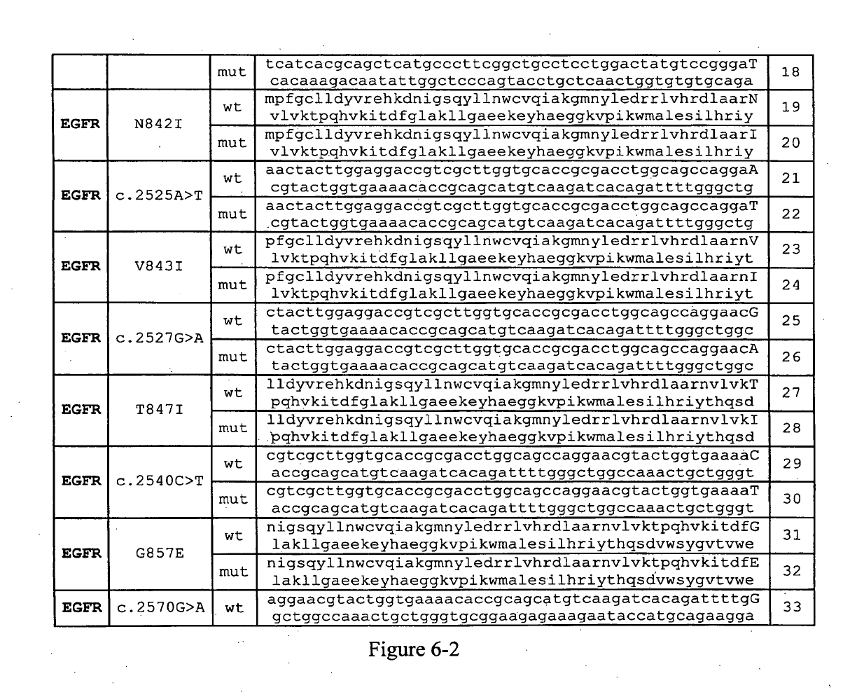

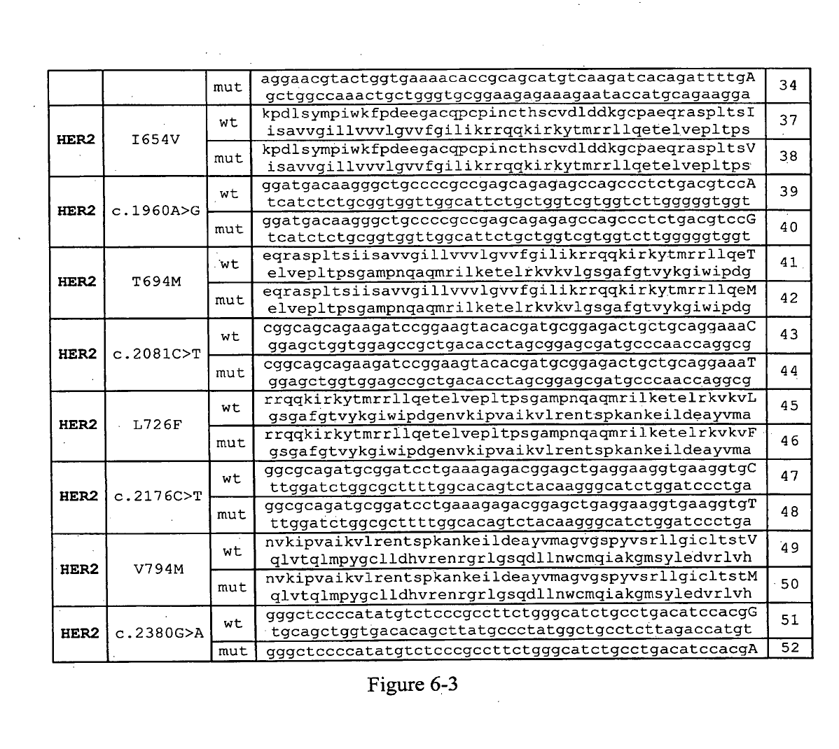

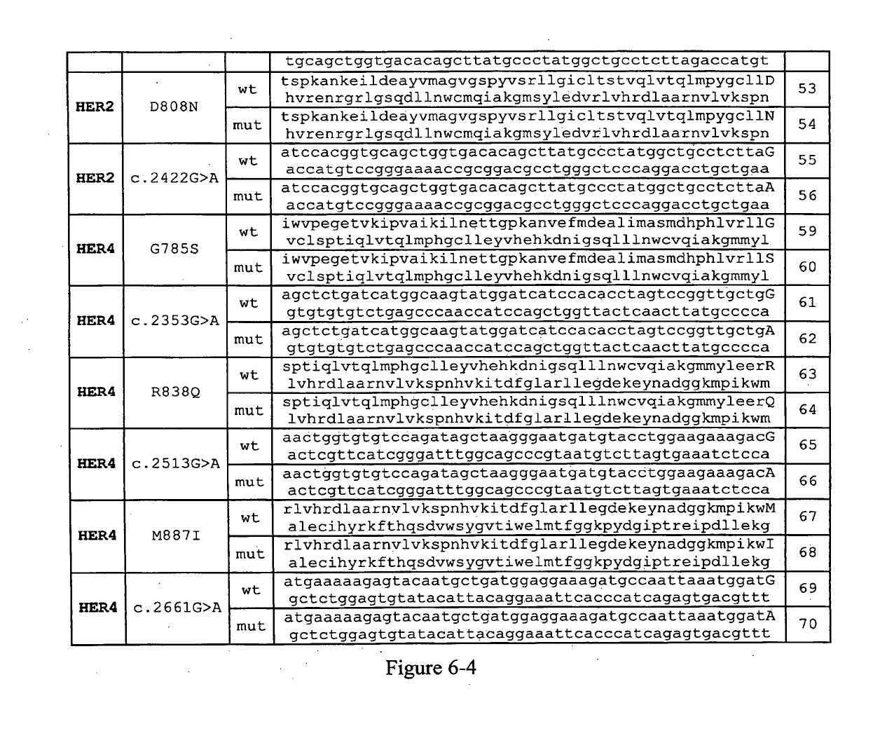

*These variants are depicted in the larger context of their respective genes and proteins in FIG. 6, with the variant amino acid or nucleotide residue in capital letters, and in the Sequence Listing. While the variant positions are given here in relation to the listed RefSeq cDNA sequences, those skilled in the art are capable of finding the corresponding variant in any alternate transcripts associated with the genes.

PTEN deficiency further adds to the predictive power as testing the status of EGFR, HER2, HER4 and PTEN allows one to accurately predict that a patient with any of activated EGFR, HER2 or HER4 or PTEN deficiency will show weak or no response to trastuzumab. Thus, the present invention concerns diagnostic, prognostic, and therapeutic methods and compositions for cancers that involve HER2 amplification/overexpression, and consequently, HER2-targeting agents.

It is contemplated that methods and compositions of the invention may be implemented with respect to cancer patients, particularly to patients with HER2-overexpressing cancers. It is understood that the term “HER2-overexpressing cancer” refers to a cancer whose etiology or cause is believed to be related to cancer cells that express higher levels of HER2 protein compared to noncancerous cells or cancer cells whose etiology or cause is not related to HER2 protein levels. Therefore, in some embodiments of the invention, the cancer being treated involves cancerous cells of the breast, lung, ovary, brain, gastrointestinal tract, salivary duct, endometrium, prostate, head & neck, glioma, pancreas, hepatocyte, myeloma, soft tissue sarcoma, or non-small cell lung cancer, but is not limited to such. In some embodiments the cancer is metastatic breast cancer. Thus in some embodiments the sample is not necessarily from the breast of the patient but may be taken from a site of metastasis.

One aspect of the invention provides a method of characterizing a patient's cancer comprising evaluating the status of EGFR, HER2 and HER4 in a sample from the patient (e.g., to determine whether the patient's tumor cells have an activating mutation in any one of these genes). In some embodiments the method further comprises evaluating the status of PTEN in a sample from the patient (e.g., to determine whether the patient's tumor cells have low or negative PTEN expression). In some embodiments the method further comprises determining whether HER2 is amplified/overexpressed in a sample from the patient.

The term “evaluate” is used according to its plain and ordinary meaning (e.g., “examine and judge carefully” or “consider”). As used herein, the “status” of a biomolecular marker (e.g., EGFR, HER2, HER4, PTEN, etc.) refers to the presence, absence, or extent/level of some physical, chemical, or genetic characteristic of the marker or its expression product(s). Such characteristics include, but are not limited to, sequence (including the presence, absence, or extent of mutations), expression levels (mRNA, protein, etc.), activity levels (enzymatic, protein-protein binding, protein-antibody binding, etc.), copy number, and gene regulation (promoter or enhancer element sequencing or copy number analysis, methylation analysis, miRNA analysis, etc.). These may be assayed directly (e.g., by assaying a gene's mRNA expression level) or determined indirectly (e.g., assaying the mRNA expression of a gene or genes whose expression level is correlated to the expression level of the gene of interest).

“Sample” as used herein refers to any biological specimen, including any tissue or fluid, that can be obtained from, or derived from a specimen obtained from, a human subject. Such samples include, healthy or tumor tissue, bodily fluids, waste matter (e.g., urine, stool), etc. In some embodiments a sample includes, but is not limited to a tissue biopsy or section, blood sample, lavage, swab, scrape, nipple aspirate, or other composition that may be extracted from the body and that contains cancer cells or elements derived from cancer cells (e.g., circulating nucleic acids, microvesicles, exosomes, etc.). In particular embodiments, the present invention concerns a sample that contains all or part of a tissue biopsy. In further embodiments, the sample contains all or part of a breast tissue biopsy, which may be from a bilateral biopsy or a unilateral biopsy. In some embodiments the sample is blood or any substance derived therefrom—e.g., serum or plasma.

“Abnormal status” means a marker's status in a particular sample differs from the status generally found in average samples (e.g., healthy samples). Examples include mutated, activated, elevated, decreased, present, absent, etc. An “elevated status” means that one or more of the above characteristics (e.g., expression) is higher than normal levels. Generally this means an increase in the characteristic (e.g., expression) as compared to a reference or index value. Conversely a “low status” means that one or more of the above characteristics (e.g., expression) is lower than normal levels. Generally this means a decrease in the characteristic (e.g., expression) as compared to a reference or index value. In this context, a “negative status” generally means the characteristic is absent or undetectable (e.g., the test sample is indistinguishable from a control). For example, PTEN status is negative if PTEN nucleic acid and/or protein is absent or undetectable in a sample. However, “negative PTEN status” also includes an inactivating mutation or copy number loss in PTEN.

The status of a gene can, according to the invention, be evaluated by various techniques. In some embodiments status is evaluated by determining whether one or more of the genes (EGFR, HER2, HER4, PTEN) has a mutation. Mutations may be detected by various suitable techniques, with which those skilled in the art are familiar. In some embodiments, mutations are detected by genotyping a sample from a patient at a particular locus of interest. Loci of particular interest in predicting a patient's likelihood of response to anti-HER2 receptor or kinase inhibitor therapy according to the invention include those in Table 1. Genotyping a sample can include allele-specific amplification (e.g., TaqMan™, Scorpions®, etc.) or allele-specific hybridization (e.g., microarray, in situ hybridization), melting temperature analysis, etc.) to detect mutations at loci of interest. These genotyping techniques are well-known to those of skill in the art and may be practiced without undue experimentation.

In some embodiments of the invention, mutations are detected by sequencing a transcript or genomic sequence of any gene of interest (e.g., EGFR, HER2, HER4, PTEN) and/or evaluating any modifications of such sequences. Sequencing can be done to determine whether there has been loss of heterozygosity (LOH). Alternatively, sequencing can provide information regarding the nature of any mutations in the gene of interest, such as deletions, insertions, frame-shifts, translocations, or truncations, which may result in mutations in the encoded protein. Such mutations can affect gene and/or protein expression and/or activity and thus are relevant to the claimed invention. In some embodiments the methods of the invention comprise sequencing the kinase domains of EGFR, HER2, and HER4. Examples of primers suitable for amplifying and sequencing the kinase domains of EGFR, HER2 and HER4 are given in Table 2 below.

TABLE 2

| | | | | SEQ |
|---|---|---|---|---|
| | | Forward/ | | ID |
| Gene | Exon | Reverse | Primer Sequence | NO |
| EGFR | 18 | F | gttttcccagtcacgacggtagagaaggcgtacatttgt | 71 |
| Kinase | | R | aggaaacagctatgaccattgatggaaatatacagcttgc | 72 |
| Domain | 19 | F | gttttcccagtcacgacggtaacatccacccagatcact | 73 |
| | | R | aggaaacagctatgaccattaggatgtggagatgagcag | 74 |
| | 20 | F | gttttcccagtcacgacgtcatgcgtcttcacctggaa | 75 |
| | | R | aggaaacagctatgaccattgaggatcctggctccttat | 76 |
| | 21 | F | gttttcccagtcacgacgagagcttcttcccatgatgat | 77 |
| | | R | aggaaacagctatgaccatatacagctagtgggaaggca | 78 |
| | 22 | F | gttttcccagtcacgacgtcgtaattaggtccagagtga | 79 |
| | | R | aggaaacagctatgaccatgcatgtcagaggatataatgta | 80 |
| | 23 | F | gttttcccagtcacgacgagcaagggattgtgattgttc | 81 |
| | | R | aggaaacagctatgaccatagctgtttggctaagagcag | 82 |
| | 24 | F | gttttcccagtcacgacgcttctttaagcaatgccatctt | 83 |
| | | R | aggaaacagctatgaccatcatgtgacagaacacagtgac | 84 |
| HER2 | 18 | F | gttttcccagtcacgacgtccgacttccctttccgaat | 85 |
| Kinase | | R | aggaaacagctatgaccattctttcaggatccgcatctg | 86 |
| Domain | 19 | F | gttttcccagtcacgacgaagtacacgatgcggagact | 87 |
| | | R | aggaaacagctatgaccataaacactgcctccagctctt | 88 |
| | 20 | F | gttttcccagtcacgacgacaagtaatgatctcctggaag | 89 |
| | | R | aggaaacagctatgaccataatgaagagagaccagagcc | 90 |
| | 21 | F | gttttcccagtcacgacgatggctgtggtttgtgatggt | 91 |
| | | R | aggaaacagctatgaccatagcacccatgtagaccttct | 92 |
| | 22 | F | gttttcccagtcacgacgtatgcacctgggctctttg | 93 |
| | | R | aggaaacagctatgaccatgtectccaactgtgtgttgt | 94 |
| | 23 | F | gttttcccagtcacgacggacagagtaccatgcagatg | 95 |
| | | R | aggaaacagctatgaccataatcctgggaagtgcacaga | 96 |
| | 24 | F | gttttcccagtcacgacgcatgatgctagactcctgag | 97 |
| | | R | aggaaacagctatgaccatgtctacatacatcctggtcc | 98 |
| HER4 | 18 | F | gttttcccagtcacgacgggcaaaccaagttggtgtgt | 99 |
| Kinase | | R | aggaaacagctatgaccatggttgtctaaagtaataactcc | 100 |
| Domain | 19 | F | gttttcccagtcacgacgtgtaacatgtaacaggtgctaa | 101 |
| | | R | aggaaacagctatgaccatatttgtaagttgtggagtttgg | 102 |
| | 20 | F | gttttcccagtcacgacgccattagtacaatccaagtaac | 103 |
| | | R | aggaaacagctatgaccataactgttccaggttaggaaata | 104 |
| | 21 | F | gttttcccagtcacgacgccaactgaaggctaagaaactt | 105 |
| | | R | aggaaacagctatgaccatcaggcttattggtttcttgtat | 106 |
| | 22 | F | gttttcccagtcacgacgcagcccaaagactcacattta | 107 |
| | | R | aggaaacagctatgaccatggaaattaggcttatcaatagg | 108 |
| | 23 | F | gttttcccagtcacgacgtagtgctggtttgttcaacata | 109 |
| | | R | aggaaacagctatgaccatcagattgagtaatctctgctat | 110 |
| | 24 | F | gttttcccagtcacgacgctttctttctcagatcattacg | 111 |
| | | R | aggaaacagctatgaccataacatgtttgtggtcctttcca | 112 |

Other methods for genetic screening may be used within the scope of the present invention, for example, to detect mutations in genomic DNA, cDNA and/or RNA samples. Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCRTM (see above), single-strand conformation polymorphism analysis (“SSCP”) and other methods well known in the art. Other methods involve silver, chromogenic or fluorescent in situ hybridization (SISH/CISH/FISH), which vividly paints chromosomes or portions of chromosomes with silver, chromogenic or fluorescent molecules. Such techniques are well known to those of skill in the art. See, e.g., Weier et al., EXPERT REV. MOL. DIAGN. (2002) 2:109-119; Moter et al., J. MICROBIOL. METHODS (2000) 41:85-112; Nath et al., BIOTECH. HISTOCHEM. (1998) 73:6-22. Another method that may also be employed involves RNA in situ hybridization (RISH). This technique may utilize nonradioactive probes such as digoxigenin-labeled copy RNA (cRNA) probes for the examination of mRNA expression, and is well known to one of ordinary skill in the art.

Those skilled in the art, apprised of the present disclosure, will be familiar with sequence analysis techniques for determining whether a variant listed in Table 1 is present in a particular nucleic acid or polypeptide—e.g., whether a serine amino acid in a test polypeptide “corresponds” to the polymorphic serine at position 50 of SEQ ID NO:2 or whether an adenine nucleotide residue in a test nucleic acid “corresponds” to the polymorphic adenine at position 50 of SEQ ID NO:4. Briefly, such techniques may include, but are not limited to: aligning the test sequence against one or more known gene sequences (e.g., EGFR cDNA sequence of SEQ ID NO:1); determining whether the test sequence has enough identity to one of these sequences to be the gene (e.g., EGFR) or a portion thereof (e.g., perfect alignment along a significant stretch or high enough percent identity to be recognized by those skilled in the art as, e.g., EGFR or a portion or variant thereof); finding a nucleotide position in the test sequence that corresponds to one of the positions listed in Table 1; and determining whether the test sequence has the variant residue listed in Table 1 for that position.

For the purpose of comparing two different nucleic acid or polypeptide sequences, one sequence (test sequence) may be described to be a specific “percentage identical to” another sequence (comparison sequence) in the present disclosure. In this respect, the percentage identity is determined by the algorithm of Karlin and Altschul, PROC. NATL. ACAD. SCI. USA, 90:5873-5877 (1993), which is incorporated into various BLAST programs. Specifically, the percentage identity is determined by the “BLAST 2 Sequences” tool, which is available at NCBI's website. See Tatusova and Madden, FEMS MICROBIOL. LETT., 174(2):247-250 (1999). For pairwise DNA-DNA comparison, the BLASTN 2.1.2 program is used with default parameters (Match: 1; Mismatch: −2; Open gap: 5 penalties; extension gap: 2 penalties; gap x_dropoff: 50; expect: 10; and word size: 11, with filter). For pairwise protein-protein sequence comparison, the BLASTP 2.1.2 program is employed using default parameters (Matrix: BLOSUM62; gap open: 11; gap extension: 1; x_dropoff: 15; expect: 10.0; and wordsize: 3, with filter). Percent identity of two sequences is calculated by aligning a test sequence with a comparison sequence using BLAST 2.1.2., determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence. When BLAST 2.1.2 is used to compare two sequences, it aligns the sequences and yields the percent identity over defined, aligned regions. If the two sequences are aligned across their entire length, the percent identity yielded by the BLAST 2.1.1 is the percent identity of the two sequences. If BLAST 2.1.2 does not align the two sequences over their entire length, then the number of identical amino acids or nucleotides in the unaligned regions of the test sequence and comparison sequence is considered to be zero and the percent identity is calculated by adding the number of identical amino acids or nucleotides in the aligned regions and dividing that number by the length of the comparison sequence.

Alternative methods for detection of deletion, insertion or substitution mutations that may be used in the practice of the present invention are disclosed in U.S. Pat. Nos. 5,849,483, 5,851,770, 5,866,337, 5,925,525 and 5,928,870, each of which is incorporated herein by reference in its entirety.

In other embodiments status is evaluated by determining the expression level of a product of one or more of the genes (e.g., mRNA, protein). Methods of the invention that involve evaluating the expression of a gene (or its product) in cancer cells can be achieved by a number of ways that directly or indirectly provide information regarding expression of the gene. Thus, ways of evaluating expression include, but are not limited to, assessing or measuring the level (including the presence or absence) of a protein, assessing or measuring the level (including the presence or absence) of a transcript, measuring a gene's copy number, etc.

Expression levels may be determined by any suitable technique. In some embodiments, expression (e.g., PTEN and/or HER2 expression) is evaluated by assessing protein levels in a sample obtained from a patient. An antibody against the protein of interest can be used in some cases to assess protein levels. Such techniques may involve using immunohistochemistry (IHC), Western blotting, ELISA, immunoprecipitation, or an antibody array. In particular embodiments, PTEN and/or HER2 protein is assessed using IHC. In some embodiments, expression is evaluated by assessing transcription. Transcription can be assessed by a variety of methods including those that involve amplifying transcripts or performing Northern blotting on transcripts. Amplification of transcripts of interest can be utilized in qPCR (including TaqMan™), which is well known to those of ordinary skill in the art. Alternatively, nuclease protection assays may be implemented to quantify transcripts. Other techniques that take advantage of hybridization between a probe and target are also contemplated, such as FISH, RISH, and microarray-based quantitation of mRNA (or mRNA-derived cDNA).

As used herein in the context of biomarkers and their expression, the “level” of a biomarker in a sample has its conventional meaning in the art. “Determining a level” herein includes quantitative determinations—e.g., mg/mL, fold change, etc. “Determining a level” herein also includes qualitative determinations—e.g., determining the presence or absence of a marker or determining whether the level of the marker is “high,” “low” or even “present” relative to some index value.

Those skilled in the art will appreciate how to obtain and use an index value in the methods of the invention. For example, the index value may represent the gene expression levels found in a normal sample obtained from the patient of interest, in which case an expression level in the tumor sample significantly higher than this index value would indicate a poor prognosis. As used herein, “index value” and “reference value” are synonymous and used interchangeably.

Alternatively the index value may represent the average expression level of a particular gene marker in a plurality of training patients (e.g., breast cancer patients) with similar outcomes whose clinical and follow-up data are available and sufficient to define and categorize the patients by disease character, e.g., response or resistance to anti-HER2 receptor therapy. See Example 1 below. For example, a “response index value” can be generated from a plurality of training cancer patients characterized as responding to anti-HER2 receptor therapy, e.g., by RECIST response criteria. A “resistance index value” can be generated from a plurality of training cancer patients defined as not responding to anti-HER2 receptor therapy, e.g., by RECIST response criteria. Thus, a response index value of a particular gene (e.g., PTEN) may represent the average level of expression of the particular gene in patients responding to treatment, whereas a resistance index value of a particular gene may represent the average level of expression of the particular gene in patients not responding to anti-HER2 receptor therapy.

In some embodiments of the invention discussed below the methods comprise determining the expression of a gene of interest (e.g., PTEN) and, if this expression is “low” or “negative,” the patient has a low or decreased likelihood of response to anti-HER2 receptor therapy. In the context of the invention and in view of the discussion of index values above, “low” expression of a relevant gene marker can mean the patient's expression level is decreased below a normal index value (e.g., by at least some threshold amount), closer to the “resistance index value” than to the “response index value,” or undetectable. Thus, when the determined level of expression of a relevant gene marker is decreased below a normal index value or more similar to the resistance index value of the gene than to the response index value of the gene, then it can be concluded that the patient has a “low likelihood of response.” On the other hand, if the determined level of expression of a relevant gene marker is at (or in the case of PTEN at or above) a normal index value or more similar to the response index value of the gene than to the resistance index value of the gene, then it can be concluded that the patient has a “high likelihood of response.” In some embodiments of the invention low or negative PTEN expression combined with the absence of an activating mutation in each of EGFR, HER2, and HER4 (and often amplification/overexpression of HER2) indicate a patient's likelihood of response is “high.”

In some embodiments of the invention, a score is assigned to a sample based on certain criteria (e.g., based on comparison to an index value as determined above), and numbers within or below a certain number or range are deemed “low.” In some embodiments, EGFR, HER2, HER4, or PTEN expression is considered below normal if an assay indicates that a particular measurement, amount or level is at about or at most about 80%, 75%, 70%, 65%, 60%, 55%, 50%, 45%, 40%, 35%, 30%, 25%, 20%, 15%, 10%, 5% or less of a reference or index amount or level. For example, a reference or index amount or level of transcript (or protein) expression may be x and a sample from the patient being tested may show an expression level of 0.5x, in which case, in some embodiments that patient may be considered to have a low level of transcript (or protein) and thus a low level of expression (i.e., “low status”). Alternatively, in some embodiments, expression is considered low if an assay indicates that a particular measurement, amount or level is about or at least about 1, 2, 3, 4, 5, 6, 7, 8, 9, 10 or more standard deviations below a reference or index amount or level. In other cases, expression may be considered low if a measurement, amount or level indicative of expression is or is at most 2, 3, 4, 5, 6, 7, 8, 9, 10, 15, 20, 25, 30, 35, 40, 45, 50 or more times less than a reference or index measurement, amount, or level.

For example, the use of IHC allows for quantitation and characterization of a protein of interest. In some embodiments IHC is used to generate an immunoreactive score (IRS) for the sample of interest. In some embodiments an IRS may be a number that is calculated based on a scale reflecting the percentage of cells staining positively for the protein(s) of interest (on a scale of 1-4, where 0=0%, 1=<10%, 2=10%-50%, 3=>50%-80%, and 4=>80%) multiplied by the intensity of staining (on a scale of 1-3, where 1=weak, 2=moderate, and 3=strong). In such embodiments IRS may range from 0-12, with high, normal or low scores either being predetermined mathematically (e.g., 1-4 are low, 5-8 are normal, and 9-12 are high) or being determined by an index value as discussed above.

In other embodiments status is evaluated using genomic copy number analysis. Cancer cells often have a different number of copies of a particular gene as compared to normal healthy cells. This copy number variation (CNV) can be deletion of one or both of the copies expected in the normal diploid cell or amplification of the gene to more than two copies. In some embodiments CNV is evaluated using FISH. In other embodiments CNV is evaluated using microarray-based techniques. Those skilled in the art are familiar with these and various other techniques for evaluating CNV.

In some embodiments status is evaluated using methylation analysis. Regulatory elements in genes can have varying levels of methylation that result in varying levels of transcription. Those skilled in the art are familiar with techniques for evaluating methylation for any given gene.

In some embodiments status is evaluated by assessing protein activity. For example, PTEN is a phosphatase and its activity can be observed in any of various techniques known to those skilled in the art. PTEN status can be evaluated, e.g., using a phosphatase assay involving a PTEN substrate, such as PIP3, or measured indirectly by measuring Akt phosphorylation. Thus, the phosphorylation level of Akt can be determined or analyzed. Alternatively, when the level of PTEN activity is down (i.e., PTEN status is low), the level of the lipid PIP3 is relatively elevated. Thus, PTEN activity can be assayed by measuring the level of PIP3. Any other compound affected by PTEN activity can be evaluated as a way of assaying for PTEN activity. EGFR, HER2, and HER4 are all kinases whose activity can be observed by techniques well-known to those skilled in the art. Phosphorylation of downstream molecules by these proteins signals the cell to proliferate, sometimes in an uncontrolled (i.e., cancerous) way. Thus measuring relatively high kinase activity by one or more of these proteins can indicate a “high” status for the protein (and by extension for the gene encoding it).

As detailed in Example 1 below, it has been discovered that evaluating the status of EGFR, HER2, and HER4 (and optionally PTEN) in a sample obtained from a patient can accurately predict whether such a patient will respond to anti-HER2 receptor or kinase inhibitor therapy. More specifically, activated status for any of EGFR, HER2 or HER4 or low (or negative) status for PTEN is highly correlated with a low likelihood (or lack) of response to anti-HER2 receptor therapy.

“Anti-HER2 therapy” means any therapeutic intervention that comprises an anti-HER2 agent. “Anti-HER2 agent” means any therapeutic agent that can directly or indirectly affect (e.g., reduce, inhibit, eliminate, or ameliorate) HER2 expression and/or activity in a cell (e.g., in a patient or in a patient's tumor). Such agents may work by directly affecting HER2 activity or they may work indirectly by affecting HER2 transcription, translation, post-translational modification, transcript or protein stability, transcript or protein localization, or some other mechanism that ultimately affects the amount of a protein's activity. Anti-HER2 therapies can be divided into two main categories: anti-HER2 receptor therapies and Anti-HER2 kinase therapies.

“Anti-HER2 receptor therapy” means any therapeutic intervention that comprises an anti-HER2 receptor agent. “Anti-HER2 receptor agent” means any therapeutic agent that acts on the extracellular domain of the HER2 protein. Examples include monoclonal antibodies targeted to the extracellular domain of HER2, e.g., trastuzumab (Herceptin™, pertuzumab (Omnitarg™), etc.

“Kinase inhibitor therapy” (“KI therapy”) means any therapeutic intervention that comprises a kinase inhibitor. “Kinase inhibitor” (“KI”) means any therapeutic agent that acts on the kinase domain of a protein kinase. Examples include gefitinib (Iressa™), erlotinib (Tarceva™), lapatinib (Tykerb™), neratinib, sorafenib, dasatinib, sunitinib, etc. “Anti-HER2 kinase therapy” means any therapeutic intervention that comprises an anti-HER2 kinase agent. “Anti-HER2 kinase agent” means a KI that acts on the kinase domain of HER2. Anti-HER2 kinase agents may act exclusively on the kinase domain of HER2 or on the kinase domain of multiple protein kinases including HER2. Examples include lapatinib (Tykerb™), neratinib, etc.

“Respond” (i.e., to “respond to” a particular therapy) has the conventional meaning one skilled in the art would give in the context of disease condition and specific therapy. In cancer, various well-defined measures of response have been devised and are well understood by those skilled in the art. For example, some objective criteria of response include Response Evaluation Criteria In Solid Tumors (RECIST), a set of published rules (e.g., changes in tumor size, etc.) that define when cancer patients improve (“respond”), stay the same (“stabilize”), or worsen (“progression”) during treatments. See, e.g., Eisenhauer et al., EUR. J. CANCER (2009) 45:228-247. “Response” can also include such metrics as “disease-free survival” (DFS), “overall survival” (OS), etc.

“Low likelihood” (or a “reduced likelihood”) of response to a particular treatment also has its conventional meaning. Often when a patient with some particular characteristic (e.g., activated status for any of EGFR, HER2 or HER4 or low status for PTEN) has a low likelihood of response to a particular treatment, this means the patient's probability of response to the treatment is lower than the probability of response for a reference population (e.g., all patients receiving the treatment, all patients receiving the treatment except those with the particular characteristic, etc.). Usually the probability of response for the patient of interest is at least a certain amount (or threshold) lower than average. For example, if a reference population of patients receiving anti-HER2 receptor therapy (e.g., trastuzumab) responds to treatment at a rate of 30%, patients with a low likelihood of response might respond at a rate of 10%. A reference population can be all patients receiving anti-HER2 receptor therapy, all patients having or not having a particular characteristic (e.g., patients having none of activated EGFR, HER2 or HER4 or low PTEN) and receiving anti-HER2 receptor therapy, etc. In some embodiments, a patient with a particular characteristic of interest (e.g., activated EGFR, HER2 or HER4 or low PTEN) has a low likelihood of response when the ratio (% R_{RP}/% R_{TP}) of the rate of response for patients with the particular characteristic of interest (% R_{RP}) to the rate of response for the reference population (% R_{RP}) is 95%, 90%, 85%, 80%, 75%, 70%, 65%, 60%, 55%, 50%, 45%, 40%, 35%, 30%, 25%, 20%, 15%, 10%, 5% or less. In other embodiments, a patient with a particular characteristic of interest (e.g., activated EGFR, HER2 or HER4 or low PTEN) has a low likelihood of response when the rate of response for patients with the particular characteristic of interest (% R_{TP}) is at least 1.5, 2, 3, 4, 5, 6, 7, 8, 9, 10, 15, 20, 25, 30, 35, 40, 45, 50 or more fold less than the rate of response for the reference population (% R_{RP}).

“Activated status” means increased activity by the encoded protein or anything that leads to such increased activity. Thus activated status can mean increased or constitutive activity in the encoded protein (e.g., caused by mutations in the encoding gene), increased expression of the encoded protein (e.g., caused by mutations in the encoding gene), increased genomic copy number, increased mRNA expression, etc. For example, activated status for EGFR and HER4 can mean amplification of the gene in the genome of a patient's tumor, increased expression (e.g., as measured by IHC) and/or a specific mutation leading to an overactive protein (See, e.g., Table 1). In the case of HER2, as used herein, “activated status” refers to activation that does not include increased gene expression (e.g., due to gene amplification). Generally “activated status” for HER2 refers to an activating mutation (e.g., a mutation that results in constitutive HER2 signaling activity such as a kinase domain mutation). This is because, in the case of HER2, activation by overexpression (e.g., due to gene amplification) actually indicates response to anti-HER2 receptor therapy (e.g., trastuzumab).

Thus one aspect of the invention provides a method of determining whether a patient will respond to anti-HER2 receptor therapy comprising evaluating EGFR, HER2 and HER4 status in a sample from the patient, wherein an activated status for any of EGFR, HER2 or HER4 indicates the patient has a reduced or low likelihood of responding to the anti-HER2 receptor therapy. Status can be evaluated by any technique known in the art, including but not limited to those techniques discussed above. In some embodiments status is evaluated by determining whether the sample has a mutation in EGFR, HER2 and/or HER4 at a particular locus (e.g., the loci shown in Table 1). In some embodiments status is evaluated by expression analysis (e.g., transcript expression, protein expression). In some embodiments status is evaluated by copy number analysis.

All genes need not be evaluated using the same technique. For instance, EGFR and HER4 status may be evaluated by genotyping while HER2 status is determined by expression analysis (e.g., IHC). Alternatively, EGFR, HER2 and HER4 can all be genotyped for activating mutations while HER2 is also evaluated for amplification (e.g., FISH) and/or over-expression (e.g., IHC). Any other combination is suitable for use in the methods of the invention.

In some embodiments the method further comprises evaluating the status of PTEN in a sample from the patient, wherein activated status for any of EGFR, HER2 or HER4 or low or negative status for PTEN indicates the patient has a reduced or low likelihood of responding to the anti-HER2 receptor therapy. PTEN status can be evaluated by any technique known in the art, including but not limited to those techniques discussed above.

Thus in some embodiments the invention provides a method of determining whether a patient will respond to anti-HER2 receptor therapy comprising (1) evaluating EGFR, HER2 and HER4 status in a sample from the patient and (2) evaluating the status of PTEN in a sample from the patient; wherein any one of (a) activated status for any of EGFR, HER2 or HER4, or (b) low or negative status for PTEN indicates the patient has a reduced or low likelihood of responding to the anti-HER2 receptor therapy. In other embodiments the invention provides a method of determining whether a patient will respond to anti-HER2 receptor therapy comprising (1) evaluating EGFR, HER2 and HER4 status in a sample from the patient and (2) evaluating the status of PTEN in a sample from the patient; wherein both of (a) no activated status for any of EGFR, HER2 or HER4 and (b) normal status for PTEN indicates the patient has a normal or increased likelihood of responding to the anti-HER2 receptor therapy.

HER2 amplification/overexpression is currently the primary gatekeeper for anti-HER2 receptor therapy—i.e., patients are only considered for, e.g., trastuzumab if their tumors show HER2 amplification/overexpression. In some embodiments the method further comprises evaluating HER2 amplification/overexpression, wherein any of (a) no HER2 amplification/overexpression, (b) activated status for any of EGFR, HER2 or HER4, or (c) low or negative status for PTEN indicates the patient has a reduced or low likelihood of responding to the anti-HER2 receptor therapy. HER2 amplification/overexpression may be evaluated by any technique known in the art, including but not limited to those techniques discussed above.

Thus in some embodiments the invention provides a method of determining whether a patient will respond to anti-HER2 receptor therapy comprising (1) evaluating whether a sample from the patient shows HER2 amplification/overexpression; (2) evaluating EGFR, HER2 and HER4 status in a sample from the patient; and (3) evaluating the status of PTEN in a sample from the patient; wherein any one of (a) no amplification/overexpression of HER2, (b) activated status for any of EGFR, HER2 or HER4, or (c) low or negative status for PTEN indicates the patient has a reduced or low likelihood of responding to the anti-HER2 receptor therapy. In other embodiments the invention provides a method of determining whether a patient will respond to anti-HER2 receptor therapy comprising (1) evaluating whether a sample from the patient shows HER2 amplification/overexpression; (2) evaluating EGFR, HER2 and HER4 status in a sample from the patient; and (3) evaluating the status of PTEN in a sample from the patient; wherein all of (a) amplification/overexpression of HER2, (b) no activated status for any of EGFR, HER2 or HER4, or (c) normal status for PTEN indicates the patient has a normal or increased likelihood of responding to the anti-HER2 receptor therapy.

In some embodiments the method further comprises evaluating some additional marker of anti-HER2 response. For example, high PIK3CA expression and/or activating mutations in PIK3CA have been associated with lack of response to trastuzumab. Thus some embodiments provide a method of determining whether a patient will respond to anti-HER2 receptor therapy comprising (1) evaluating whether a sample from the patient shows HER2 amplification/overexpression; (2) evaluating EGFR, HER2 and HER4 status in a sample from the patient; (3) evaluating the status of PTEN in a sample from the patient; and (4) evaluating PIK3CA status in a sample from the patient; wherein any one of (a) no amplification/overexpression of HER2, (b) activated status for any of EGFR, HER2 or HER4, (c) low or negative status for PTEN, or (d) activated PIK3CA status indicates the patient has a reduced or low likelihood of responding to the anti-HER2 receptor therapy. In some embodiments the methods of the invention further comprise determining the status of other additional markers that may improve the predictive power of the methods of the invention, including AKT (Entrez GeneId No. 207) and p70S6K (Entrez GeneId No. 6198). In some embodiments the status to be determined includes p-AKT-Ser473 and/or p-p70S6K-Thr389.

In Example 1 below activating mutations in the kinase domain of HER4 (as well as in EGFR and HER2) have been discovered and these mutations have been shown to exert an effect on response to drugs targeting HER2. More specifically, the presence of HER4 (and EGFR and HER2) kinase domain mutations confers resistance to trastuzumab, a monoclonal antibody targeting the extracellular domain of HER2. Thus mutations in the kinase domain of HER4 (or EGFR or HER2) will, according to the present invention, confer (and therefore predict) sensitivity to KIs (e.g., lapatinib, erlotinib, gefitinib, etc.).

Thus one aspect of the invention provides a method of determining whether a patient will respond to KI therapy comprising evaluating HER4 status in a sample from the patient, wherein an activated status (e.g., activating mutation) for HER4 indicates the patient has a high (or increased or at least not reduced) likelihood of responding to the KI therapy. In some embodiments the invention provides a method of determining whether a patient will respond to KI therapy comprising evaluating EGFR, HER2 and HER4 status in a sample from the patient, wherein an activating mutation in any of EGFR, HER2 or HER4 indicates the patient has a high (or increased or at least not reduced) likelihood of responding to the KI therapy. In some embodiments the invention provides a method of determining whether a patient will respond to KI therapy comprising evaluating EGFR, HER2, HER4, and PTEN status in a sample from the patient, wherein an activating mutation in any of EGFR, HER2 or HER4 and normal status for PTEN indicates the patient has a high (or increased or at least not reduced) likelihood of responding to the KI therapy.

Yet another aspect of the invention provides a method of optimizing treatment of a cancer patient comprising evaluating EGFR, HER2 and HER4 status in a sample from the patient and recommending, prescribing or administering a treatment regimen that does not include an anti-HER2 receptor agent (e.g., a treatment regimen comprising a KI, such as lapatinib) if the sample shows an activated status for any of EGFR, HER2 or HER4. In some embodiments the treatment optimization method comprises evaluating EGFR, HER2, HER4, and PTEN status in a sample from the patient and recommending, prescribing or administering a treatment regimen that does not include an anti-HER2 receptor agent (e.g., a treatment regimen comprising a KI, such as lapatinib) if the sample shows an activated status for any of EGFR, HER2 or HER4 or a low or negative status for PTEN. Examples of “a treatment regimen that does not include an anti-HER2 receptor agent” include any combination of KI therapy (e.g., lapatinib), radiation therapy, chemotherapy (e.g., capecitabine, platinum drug such as carboplatin, cisplatin or oxaloplatin, etc.), a taxane (e.g., docetaxel, paclitaxel), hormone therapy (e.g., tamoxifen, megestrol), and/or an aromatase inhibitor (e.g., letrozole, anastrozole, exemestane). Specific examples include tamoxifen monotherapy; tamoxifen plus radiation; cyclophosphamide plus doxorubicin/Adriomycin (CA); CA plus a taxane drug, such as docetaxel (CAT); cyclophosphamide, methotrexate, and fluorouracil (CMF); etc.

In some embodiments the treatment optimization method comprises evaluating HER2 overexpression and EGFR, HER2 and HER4 status in a sample from the patient and recommending, prescribing or administering a treatment regimen that includes an anti-HER2 receptor agent if the sample shows HER2 overexpression and does not show an activated status for each of EGFR, HER2 and HER4. In some embodiments the treatment optimization method comprises evaluating HER2 overexpression and EGFR, HER2, HER4, and PTEN status in a sample from the patient and recommending, prescribing or administering a treatment regimen that includes an anti-HER2 receptor agent if the sample shows HER2 overexpression, does not show an activated status for each of EGFR, HER2 and HER4, and does not show a low or negative status for PTEN. Examples of “a treatment regimen that includes an anti-HER2 receptor agent” include trastuzumab or pertuzumab monotherapy or trastuzumab or pertuzumab plus any combination of KI therapy (e.g., lapatinib, erlotinib, gefitinib, etc.), radiation therapy, chemotherapy (e.g., capecitabine, platinum drug such as carboplatin, cisplatin or oxaloplatin, etc.), a taxane (e.g., docetaxel, paclitaxel), hormone therapy (e.g., tamoxifen, megestrol), an aromatase inhibitor (e.g., letrozole, anastrozole, exemestane), and/or HER2/neu vaccination. Specific examples include every-4-week carboplatin and weekly paclitaxel with trastuzumab; and those discussed in Murphy & Modi, BIOLOGICS (2009) 3:289-301.

In some embodiments the invention provides a method of optimizing treatment of a cancer patient comprising evaluating EGFR, HER2 and HER4 status in a sample from the patient and either (a) recommending, prescribing or administering a treatment regimen that does not include an anti-HER2 receptor agent (e.g., a treatment regimen comprising a KI, such as lapatinib) if the sample shows an activated status for any of EGFR, HER2 or HER4 or (b) recommending, prescribing or administering a treatment regimen comprising an anti-HER2 receptor agent (e.g., a treatment regimen comprising trastuzumab) if the sample does not show an activated status for any of EGFR, HER2 or HER4. Thus some embodiments provide a method of optimizing treatment of a cancer patient comprising evaluating EGFR, HER2 and HER4 status in a sample from the patient and either (a) recommending, prescribing or administering a treatment regimen that comprising lapatinib and not comprising trastuzumab if the sample shows an activated status for any of EGFR, HER2 or HER4 or (b) recommending, prescribing or administering a treatment regimen comprising trastuzumab if the sample does not show an activated status for any of EGFR, HER2 or HER4. In some embodiments the treatment optimization method comprises evaluating EGFR, HER2, HER4, and PTEN status in a sample from the patient and either (a) recommending, prescribing or administering a treatment regimen that does not include an anti-HER2 receptor agent (e.g., a treatment regimen comprising a KI, such as lapatinib) if the sample shows an activated status for any of EGFR, HER2 or HER4 or a low or negative status for PTEN or (b) recommending, prescribing or administering a treatment regimen comprising an anti-HER2 receptor agent (e.g., a treatment regimen comprising trastuzumab) if the sample does not show an activated status for any of EGFR, HER2 or HER4 or does not show low or negative PTEN status.

In some embodiments of this and other aspects of the invention the patient is a breast cancer patient. In some embodiments the patient is an estrogen receptor negative patient (i.e., the patient's tumor is estrogen receptor negative). In some embodiments the patient is a progesterone receptor negative patient (i.e., the patient's tumor is progesterone receptor negative).

In some embodiments the treatment optimization method is implemented on a computer. Thus the invention provides a computer-implemented method of optimizing treatment of a cancer patient comprising: accessing status information for EGFR, HER2 and HER4 derived from a patient sample and stored in a computer-readable medium; querying this information to determine whether the patient has an activated status for any of these genes; outputting [or displaying] the likelihood of the patient responding to anti-HER2 receptor therapy and/or KI therapy based on the status of these genes. In some embodiments the method may end by additionally or alternatively giving some recommendation as to whether the patient should receive anti-HER2 receptor therapy (e.g., recommending no anti-HER2 receptor therapy if any one of EGFR, HER2 or HER4 is activated) or KI therapy (e.g., recommending KI therapy if none of EGFR, HER2 or HER4 is activated). In some embodiments an algorithm is used to calculate the likelihood of the patient responding to anti-HER2 receptor or KI therapy based the status of EGFR, HER2, HER4, and optionally PTEN (along with any additional markers).

As used herein in the context of computer-implemented embodiments of the invention, “displaying” means communicating any information by any sensory means. Examples include, but are not limited to, visual displays, e.g., on a computer screen or on a sheet of paper printed at the command of the computer, and auditory displays, e.g., computer generated or recorded auditory expression of a patient's genotype.

Still another aspect of the invention provides apparatus and systems for determining whether a patient will respond to anti-HER2 receptor or KI therapy. In some embodiments the systems of the present invention will include (e.g., be programmed to and capable of performing) computer-implemented methods of the invention. Generally speaking, the system comprises (1) computer means for receiving and/or storing gene status data (e.g., mutation status, expression level, activity level); (2) computer means for identifying a patient with activated status for any of EGFR, HER2 or HER4 or low or negative status for PTEN; and (3) computer means for concluding whether there is a low or decreased likelihood that the patient will respond to anti-HER2 receptor therapy or whether there is a normal or increase likelihood that the patient will respond to KI therapy. In some embodiments the system may additionally comprise a computer means for communicating (e.g., informing a health care professional) (a) that the patient has an activated status for any of EGFR, HER2 or HER4 or low or negative status for PTEN and/or (b) there is a low or decreased likelihood that the patient will respond to anti-HER2 receptor therapy if (a) is true; or (c) that the patient does not have an activated status for any of EGFR, HER2 or HER4 or low or negative status for PTEN and/or (d) there is a normal or increased likelihood that the patient will respond to KI therapy if (c) is true.

In one embodiment the invention provides a system for determining whether a patient will respond to anti-HER2 receptor or KI therapy, comprising: (1) a sample analyzer for determining the status of EGFR, HER2, HER4, and optionally PTEN, wherein the sample analyzer contains the sample or biomolecules from the sample (e.g., DNA, RNA, protein); (2) a first computer program means for (a) receiving status data on EGFR, HER2, HER4, and optionally PTEN, and (b) determining, based on such status data, whether the patient will respond to anti-HER2 receptor or KI therapy. In some embodiments the computer program means determines that the patient will not respond to anti-HER2 receptor or KI therapy if the status data indicates any of the following: activated EGFR status, activated HER4 status, or an activated HER2 status (or optionally loss of PTEN).

In one embodiment the invention provides a system for determining whether a patient will respond to anti-HER2 receptor or KI therapy, comprising: (1) a sample analyzer for determining the status of EGFR, HER2, HER4, and optionally PTEN, wherein the sample analyzer contains the sample or biomolecules from the sample (e.g., DNA, RNA, protein); (2) a first computer program means for (a) receiving status data on EGFR, HER2, HER4, and optionally PTEN, and (b) combining the determined status of each of EGFR, HER2, HER4, and optionally PTEN, to provide a test value; and optionally (3) a second computer program means for comparing the test value to one or more reference values each associated with a predetermined degree of probability of response to anti-HER2 receptor or KI therapy.

In some embodiments, the system further comprises a display module displaying the comparison between the test value and the one or more reference values, or displaying a result of the comparing step.

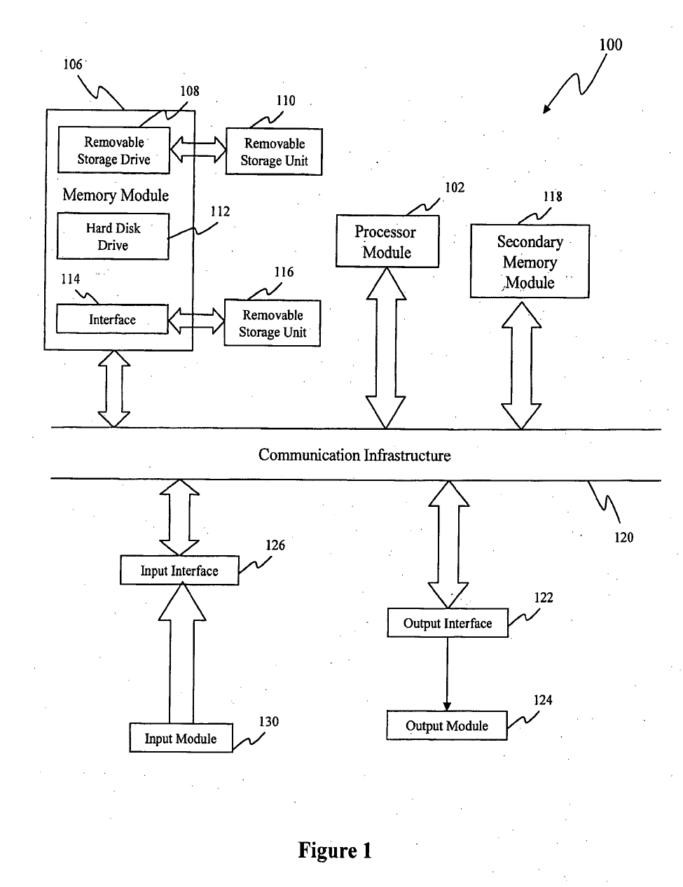

One example of such a system is the computer system [100] illustrated in FIG. 1. Such a computer system [100] may include at least one input module [130] for entering patient data into the computer system [100]. The computer system [100] may include at least one output module [124] for indicating (a) that the patient has an activated status for any of EGFR, HER2 or HER4 or low or negative status for PTEN; (b) there is a low or decreased likelihood that the patient will respond to anti-HER2 receptor therapy if (a) is true; and/or (c) suggested treatments (e.g., KI therapy such as lapatinib) determined by the computer system [100] if (a) is true. The computer system [100] may include at least one memory module [106] in communication with the at least one input module [130] and the at least one output module [124], the memory module being capable, inter alia, of storing patient gene status data and/or conclusions regarding likelihood of response to anti-HER2 receptor therapy.

The at least one memory module [106] may include, e.g., a removable storage drive [108], which can be in various forms, including but not limited to, a magnetic tape drive, a floppy disk drive, a VCD drive, a DVD drive, an optical disk drive, etc. The removable storage drive [108] may be compatible with a removable storage unit [110] such that it can read from and/or write to the removable storage unit [110]. The removable storage unit [110] may include a computer usable storage medium having stored therein computer-readable program codes or instructions and/or computer-readable data. For example, the removable storage unit [110] may store patient data. Examples of removable storage units [110] are well known in the art, including, but not limited to, floppy disks, magnetic tapes, optical disks, flash memory drives, and the like. The at least one memory module [106] may also include a hard disk drive [112], which can be used to store computer-readable program codes or instructions, and/or computer-readable data.

In addition, as shown in FIG. 1, the at least one memory module [106] may further include an interface [114] and a removable storage unit [116] that is compatible with the interface [114] such that software, computer-readable codes or instructions can be transferred from the removable storage unit [116] into the computer system [100]. Examples of interface [114] and removable storage unit [116] pairs include, e.g., removable memory chips (e.g., EPROMs or PROMs) and sockets associated therewith, program cartridges and cartridge interface, and the like. The computer system [100] may also include a secondary memory module [118], such as random access memory (RAM).

The computer system [100] may include at least one processor module [102]. It should be understood that the at least one processor module [102] may consist of any number of devices. The at least one processor module [102] may include a data processing device, such as a microprocessor or microcontroller or a central processing unit. The at least one processor module [102] may include another logic device such as a DMA (Direct Memory Access) processor, an integrated communication processor device, a custom VLSI (Very Large Scale Integration) device or an ASIC (Application Specific Integrated Circuit) device. In addition, the at least one processor module [102] may include any other type of analog or digital circuitry that is designed to perform the processing functions described herein.

As shown in FIG. 1, in the computer system [100], the at least one memory module [106], the at least one processor module [102], and secondary memory module [118] are all operably linked together through a communication infrastructure [120], which may be a communications bus, system board, cross-bar, etc. Through the communication infrastructure [120], computer program codes or instructions or computer-readable data can be transferred and exchanged. An input interface [126] may operably connect the at least one input module [126] to the communication infrastructure [120]. Likewise, an output interface [122] may operably connect the at least one output module [124] to the communication infrastructure [120].

The at least one input module [130] may include, for example, a keyboard, mouse, touch screen, scanner, and other input devices known in the art. The at least one output module [124] may include, for example, a display screen, such as a computer monitor, TV monitor, or the touch screen of the at least one input module [130]; a printer; and audio speakers. The computer system [100] may also include, modems, communication ports, network cards such as Ethernet cards, and newly developed devices for accessing intranets or the Internet.

The at least one memory module [106] may be configured for storing patient data received an intranet or the Internet or entered via the at least one input module [130] and processed via the at least one processor module [102]. Patient data relevant to the present invention may include expression level, activity level, and/or sequence information for EGFR, HER2, HER4, PTEN, and/or any additional markers. Any other patient data a physician might find useful in making treatment decisions/recommendations may also be entered into the system, including but not limited to age, gender, and race/ethnicity and lifestyle data such as diet information. Other possible types of patient data include symptoms currently or previously experienced, patient's history of illnesses, patient's family medical history, medications, and medical procedures.

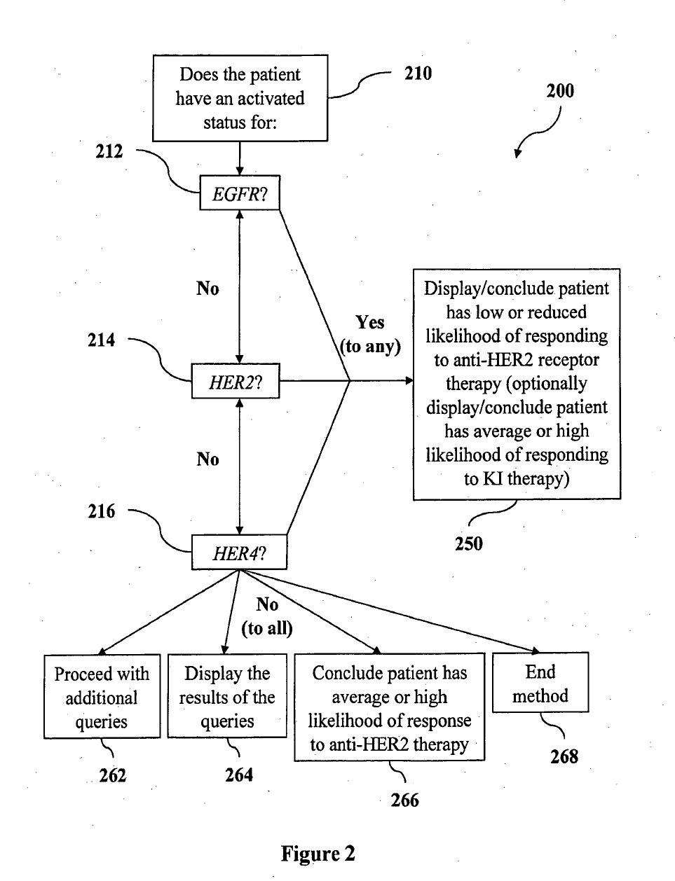

The at least one memory module [106] may include a computer-implemented method stored therein. The at least one processor module [102] may be used to execute software or computer-readable instruction codes of the computer-implemented method. The computer-implemented method may be configured to, based upon the patient data, indicate an average, increased or decreased likelihood of response to anti-HER2 receptor therapy, generate a list of possible treatments (e.g., lapatinib), etc. The above systems may be embodied in apparatus of the invention, which are special purpose computers when programmed (as by installation of software) to perform the methods ([200], [300], [400], [500]) illustrated in FIGS. 2-5.

In certain embodiments, the computer-implemented method may be configured to identify a patient as having either an average (or high) or a low (or reduced) likelihood of responding to anti-HER2 receptor or KI therapy. For example, the computer-implemented method may be configured to inform a physician that a particular patient has a low likelihood of responding to anti-HER2 receptor therapy. Alternatively or additionally, the computer-implemented method may be configured to actually suggest a particular course of treatment (e.g., a treatment regimen comprising a KI) based on the answers to various queries.

FIG. 2 illustrates one embodiment of a computer-implemented method [200] of the invention that may be implemented with the computer system [100] of the invention. The method may begin with the query “Does the patient have an activated status for” [210] any of EGFR [212], HER2 [214], and/or HER4 [216]. If the answer to any of these queries is “yes,” the method may Display/conclude patient has low or reduced likelihood of responding to anti-HER2 receptor therapy (optionally display/conclude patient has average or high likelihood of responding to KI therapy) [250]. Alternatively the method may simply display the results of the queries (i.e., that patient does or does not have an activated status for any or all of the genes of interest) and/or proceed with additional queries. If the answer to all of these queries is “no,” the method may proceed [262] with more queries (e.g., aimed at evaluating additional markers), display the results of the queries [264], conclude that the patient has an average or high likelihood of response to anti-HER2 receptor therapy [266], or simply end [268]. It should be noted that FIG. 2 is not intended to imply any particular order for the queries. In some embodiments, not all queries need be asked. For instance, if the answer to “Does the patients have an activated status of EGFR?” [212] is “yes,” the method may conclude the patients has a low likelihood of response [250] without needing to perform any other queries (e.g., [214] or [216]).

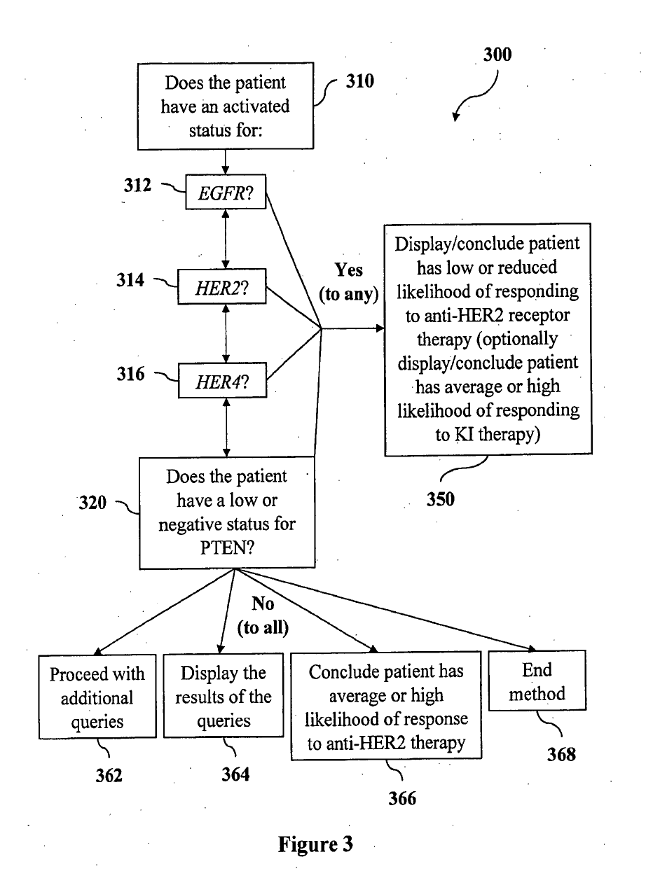

FIG. 3 illustrates another embodiment of a computer-implemented method [300] of the invention that may be implemented with the computer system [100] of the invention. The method [300] may begin with the query “Does the patient have an activated status for . . . ” [310] any of EGFR [312], HER2 [314], and/or HER4 [316]. The method [300] then performs the query “Does the patient have a low or negative status for PTEN?” [320]. If the answer to any of these queries is “yes,” the method may display/conclude patient has low or reduced likelihood of responding to anti-HER2 receptor therapy (optionally display/conclude patient has average or high likelihood of responding to KI therapy) [350]. Alternatively the method may simply display the results of the queries (i.e., that patient does or does not have an activated status for any or all of the genes of interest) and/or proceed with additional queries. If the answer to all of these queries is “no,” the method may proceed [362] with more queries (e.g., aimed at evaluating additional markers), display the results of the queries [364], conclude that the patient has an average or high likelihood of response to anti-HER2 receptor therapy [366], or simply end [368]. It should be noted that FIG. 3 is not intended to imply any particular order for the queries. In some embodiments, not all queries need be asked. For instance, if the answer to “Does the patients have an activated status of EGFR?” [312] is “yes,” the method may conclude the patients has a low likelihood of response [350] without needing to perform any other queries (e.g., [314], [316], or [320]).

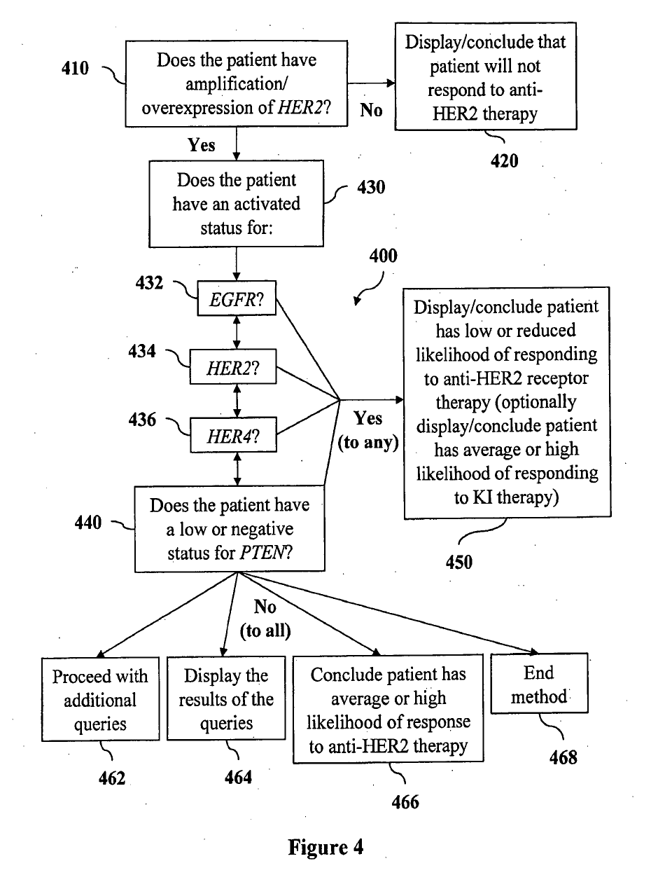

FIG. 4 illustrates yet another embodiment of a computer-implemented method [400] of the invention that may be implemented with the computer system [100] of the invention. The method [400] may begin with the query “Does the patient have amplification/overexpression of HER2?” [410]. If the answer is “no,” the method [400] may display/conclude that patient will not respond to anti-HER2 therapy [420]. If the answer is “yes,” the method [400] may proceed with the query “Does the patient have an activated status for . . . ” [430] any of EGFR [432], HER2 [434], and/or HER4 [436]. The method [400] then performs the query “Does the patient have a low or negative status for PTEN?” [440]. If the answer to any of these queries ([430], [432], [434], [436], or [440]) is “yes,” the method [400] may display/conclude patient has low or reduced likelihood of responding to anti-HER2 receptor therapy (optionally display/conclude patient has average or high likelihood of responding to KI therapy) [450]. Alternatively the method [400] may simply display the results of the queries (i.e., that patient does or does not have an activated status for any or all of the genes of interest) and/or proceed with additional queries. If the answer to all of these queries ([430], [432], [434], [436], and [440]) is “no,” the method [400] may proceed [462] with more queries (e.g., aimed at evaluating additional markers), display the results of the queries [464], conclude that the patient has an average or high likelihood of response to anti-HER2 therapy [466], or simply end [468]. It should be noted that FIG. 4 is not intended to imply any particular order for the queries. In some embodiments, not all queries need be asked. For instance, if the answer to “Does the patients have an activated status of EGFR?” [432] is “yes,” the method may conclude the patients has a low likelihood of response [450] without needing to perform any other queries (e.g., [434], [436], or [440]).

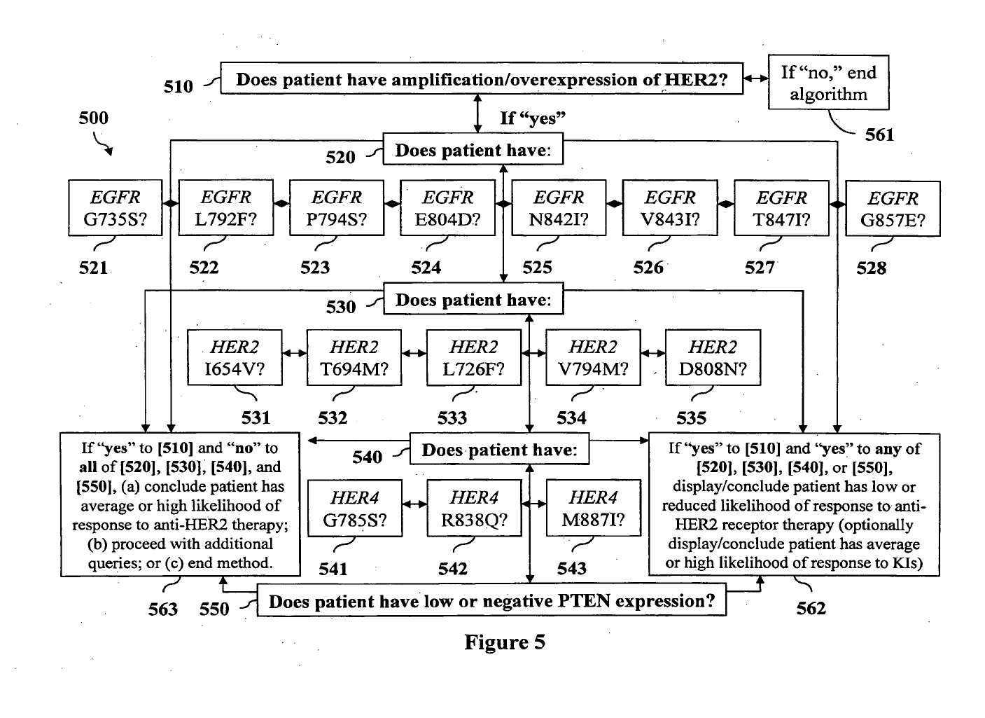

FIG. 5 illustrates yet another embodiment of a computer-implemented method [400] of the invention that may be implemented with the computer system [100] of the invention. The method [500] may begin with the query “Does the patient have amplification/overexpression of HER2?” [510]. If the answer is “no,” the method [500] may display/conclude that patient will not respond to anti-HER2 therapy [561]. If the answer is “yes,” the method [500] may proceed with the query “Does the patient have . . . ” ([520], [530] or [540]) any of the following genetic variants: EGFR G735S [521], EGFR L792F [522], EGFR P794S [523], EGFR E804D [524], EGFR N842I [525], EGFR V843I [526], EGFR T847I [527], EGFR G857E [528], HER2 I654V [531], HER2 T694M [532], HER2 L726F [533], HER2 V794M [534], HER2 D808N [535], HER4 G785S [541], HER4 R838Q [542], or HER4 M887I [543]. The method [500] may further perform the query “Does patient have low or negative PTEN expression” [550]. If the answer to [510] is “yes” and the answer to any of these further queries ([520], [521], [522], [523], [524], [525], [526], [527], [528], [530], [531], [532], [533], [534], [535], [540], [541], [542], [543], or [550]) is also “yes,” the method [500] may display/conclude the patient has low or reduced likelihood of responding to anti-HER2 receptor therapy (optionally display/conclude patient has average or high likelihood of responding to KI therapy) [562]. Alternatively the method [500] may simply display the results of the queries (i.e., that patient does or does not have an activated status for any or all of the genes of interest) and/or proceed with additional queries. If the answer to [510] is “yes” and the answer to all of these further queries ([520], [521], [522], [523], [524], [525], [526], [527], [528], [530], [531], [532], [533], [534], [535], [540], [541], [542], [543], or [550]) is “no,” the method [500] may (a) conclude that the patient has an average or high likelihood of response to anti-HER2 therapy, (b) proceed with more queries (e.g., aimed at evaluating additional markers), and/or (c) simply end [563]. Alternatively or additionally, the method [500] may display the results of the queries.

The above computer-implemented methods ([200], [300], [400], and [500]) may make the indicated queries in the order indicated above or in any other order. In some embodiments of the method [300] illustrated in FIG. 3, for example, the method asks about the status of EGFR, HER2, and/or HER4 [310] before asking about PTEN status [320]. In preferred embodiments of the methods ([400], [500]) illustrated in FIGS. 4 & 5, for example, the methods query HER2 amplification/overexpression ([410], [510]) first.

In some embodiments the method concludes ([250], [350], [450], [562]) after an answer of “yes” to any of the status queries for EGFR, HER2, HER4 and PTEN without performing any remaining status queries. In other embodiments the method concludes ([250], [350], [450], [562]) only after certain “yes” answers (e.g., “yes” to HER4 or to PTEN and to EGFR). Likewise, in some embodiments, one or more “no” answers short of querying all of the listed genes or variants (e.g., “no” to HER2 amplification/overexpression) is sufficient to either end the method or prompt additional queries (e.g., clinical parameters). In some embodiments, rather than immediately reaching a conclusion after one or more “yes” or “no” answers, the method instead proceeds with additional queries (e.g., clinical parameters). In this way, the method may be “weighted” such that the answers to some queries can outweigh or even completely override counter-indicative answers to other queries.

In some embodiments, each of the above methods of the invention [200, 300, 400, 500] is open-ended. In other words, the apparent first step [210, 310, 410, 510] in the Figures may actually form part of a larger process and, within this larger process, need not be the first step/query. Additional steps may also be added onto the minimal methods discussed above. These additional steps may include, but are not limited to, informing a health care professional (or the patient itself) of the conclusion reached according to the method; combining the conclusion reached by the illustrated method with other facts or conclusions to reach some additional or refined conclusion regarding the patient's treatment; making a recommendation for treatment (e.g., “patient should/should not be prescribed an anti-HER2 receptor therapy”); additional queries about additional biomarkers (e.g., HER2 expression level) or about other useful patient information (e.g., age at diagnosis, general patient health, clinical parameters, etc.).

Regarding the above methods [200, 300, 400, 500], the answers to the queries [210, 310, 410, 510, 512, 514] may be determined by the respective method instituting a search of patient data for the answer. For example, to answer the respective queries [210, 310, 320, 410, 430, 440, 510, 520, 530, 540, 550], patient data may be searched for mutation (i.e., sequence), expression level, activity level, and/or copy number data for EGFR, HER2, HER4, and/or PTEN. If such a comparison has not already been performed, the method may compare these data to some reference value or sequence in order to determine if the patient has, e.g., a higher or lower expression or activity level or a mutation. Additionally or alternatively, the method may present one or both of the queries [210, 310, 320, 410, 430, 440, 510, 520, 530, 540, 550] to a user of the computer system [100] (e.g., a physician) for the user's response. For example, the questions [210, 310, 320, 410, 430, 440, 510, 520, 530, 540, 550] may be presented via an output module [124]. The user may then answer “yes” or “no” via an input module [130]. The method may then proceed based upon the answer received. Likewise, the conclusions ([250], [350], [450], [562]) may be presented to a user of the respective method via an output module [124].

The results of these and any other analyses according to the invention are often communicated to physicians, genetic counselors and/or patients (or other interested parties such as researchers) in a transmittable form that can be communicated or transmitted to any of the above parties. Such a form can vary and can be tangible or intangible. The results can be embodied in descriptive statements, diagrams, photographs, charts, images or any other visual forms. For example, graphs showing expression or activity level or sequence variation information for various genes can be used in explaining the results. Diagrams showing such information for additional target gene(s) are also useful in indicating some testing results. The statements and visual forms can be recorded on a tangible medium such as papers, computer readable media such as floppy disks, compact disks, etc., or on an intangible medium, e.g., an electronic medium in the form of email or website on internet or intranet. In addition, results can also be recorded in a sound form and transmitted through any suitable medium, e.g., analog or digital cable lines, fiber optic cables, etc., via telephone, facsimile, wireless mobile phone, internet phone and the like.

Thus, the information and data on a test result can be produced anywhere in the world and transmitted to a different location. As an illustrative example, when an expression level, activity level, or sequencing (or genotyping) assay is conducted outside the United States, the information and data on a test result may be generated, cast in a transmittable form as described above, and then imported into the United States. Indeed, such information can then be incorporated into a system as described in FIG. 1 for use in methods as in FIGS. 2-5. Accordingly, the present invention also encompasses a method for producing a transmittable form of information on at least one of (a) expression level, (b) activity level, or (c) sequence variation (mutation) for at least one patient sample. The method comprises the steps of (1) determining at least one of (a), (b), or (c) above according to methods of the present invention; and (2) embodying the result of the determining step in a transmittable form. The transmittable form is the product of such a method.

Techniques for analyzing such status data (indeed any data obtained according to the invention) will often be implemented using hardware, software or a combination thereof in one or more computer systems or other processing systems capable of effectuating such analysis.

The computer-based analysis function can be implemented in any suitable language and/or browsers. For example, it may be implemented with C language and preferably using object-oriented high-level programming languages such as Visual Basic, SmallTalk, C++, and the like. The application can be written to suit environments such as the Microsoft Windows™ environment including Windows™ 98, Windows™ 2000, Windows™ NT, and the like. In addition, the application can also be written for the MacIntosh™, SUN™, UNIX or LINUX environment. In addition, the functional steps can also be implemented using a universal or platform-independent programming language. Examples of such multi-platform programming languages include, but are not limited to, hypertext markup language (HTML), JAVA™, JavaScript™, Flash programming language, common gateway interface/structured query language (CGI/SQL), practical extraction report language (PERL), AppleScript™ and other system script languages, programming language/structured query language (PL/SQL), and the like. Java™—or JavaScript™-enabled browsers such as HotJava™, Microsoft™ Explorer™, or Netscape™ can be used. When active content web pages are used, they may include Java™ applets or ActiveX™ controls or other active content technologies.

The analysis function can also be embodied in computer program products and used in the systems described above or other computer- or internet-based systems. Accordingly, another aspect of the present invention relates to a computer program product comprising a computer-usable medium having computer-readable program codes or instructions embodied thereon for enabling a processor to carry out expression, activity, or sequence analysis. These computer program instructions may be loaded onto a computer or other programmable apparatus to produce a machine, such that the instructions which execute on the computer or other programmable apparatus create means for implementing the functions or steps described above. These computer program instructions may also be stored in a computer-readable memory or medium that can direct a computer or other programmable apparatus to function in a particular manner, such that the instructions stored in the computer-readable memory or medium produce an article of manufacture including instruction means which implement the analysis. The computer program instructions may also be loaded onto a computer or other programmable apparatus to cause a series of operational steps to be performed on the computer or other programmable apparatus to produce a computer implemented process such that the instructions which execute on the computer or other programmable apparatus provide steps for implementing the functions or steps described above.

The practice of the present invention may also employ conventional biology methods, software and systems. Computer software products of the invention typically include computer readable media having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. Basic computational biology methods are described in, for example, Setubal et al., INTRODUCTION TO COMPUTATIONAL BIOLOGY METHODS (PWS Publishing Company, Boston, 1997); Salzberg et al. (Ed.), COMPUTATIONAL METHODS IN MOLECULAR BIOLOGY, (Elsevier, Amsterdam, 1998); Rashidi & Buehler, BIOINFORMATICS BASICS: APPLICATION IN BIOLOGICAL SCIENCE AND MEDICINE (CRC Press, London, 2000); and Ouelette & Bzevanis, BIOINFORMATICS: A PRACTICAL GUIDE FOR ANALYSIS OF GENE AND PROTEINS (Wiley & Sons, Inc., 2^{nd} ed., 2001); see also, U.S. Pat. No. 6,420,108.

The present invention may also make use of various computer program products and software for a variety of purposes, such as probe design, management of data, analysis, and instrument operation. See U.S. Pat. Nos. 5,593,839; 5,795,716; 5,733,729; 5,974,164; 6,066,454; 6,090,555; 6,185,561; 6,188,783; 6,223,127; 6,229,911 and 6,308,170.

Additionally, the present invention may have embodiments that include methods for providing genetic information over networks such as the Internet as shown in U.S. Ser. Nos. 10/197,621 (U.S. Pub. No. 20030097222); 10/063,559 (U.S. Pub. No. 20020183936), 10/065,856 (U.S. Pub. No. 20030100995); 10/065,868 (U.S. Pub. No. 20030120432); 10/423,403 (U.S. Pub. No. 20040049354).

Another aspect of the invention provides compositions comprising EGFR, HER2, HER4, or PTEN nucleic acids or proteins or nucleic acids or proteins targeted thereto. Thus one aspect of the invention provides isolated nucleic acids comprising at least one variant listed Table 1. As used herein, a nucleic acid or polypeptide “comprises” a variant if the nucleic acid or polypeptide contains or encompasses a residue corresponding to such variant within its linear sequence. A nucleic acid or polypeptide comprises a variant if the variant is found in any part of the linear sequence, including either end (e.g., the extreme 5′ or 3′ end in nucleic acids or the extreme N-terminal or C-terminal end in polypeptides).

The term “isolated” when used in reference to nucleic acids (e.g., genomic DNAs, cDNAs, mRNAs, or fragments thereof) is intended to mean that a nucleic acid molecule is present in a form that is substantially separated from other naturally occurring nucleic acids that are normally associated with the molecule. For example, since a naturally existing chromosome (or a viral equivalent thereof) includes a long nucleic acid sequence, an “isolated nucleic acid” as used herein means a nucleic acid molecule having only a portion of the nucleic acid sequence in the chromosome but not one or more other portions present on the same chromosome. More specifically, an “isolated nucleic acid” typically includes no more than 25 kb of naturally occurring nucleic acid sequence which immediately flanks the nucleic acid in the naturally existing chromosome (or a viral equivalent thereof). However, it is noted that an “isolated nucleic acid” as used herein is distinct from a clone in a conventional library such as genomic DNA library and cDNA library in that the clone in a library is still in admixture with almost all the other nucleic acids of a chromosome or cell. Thus, an “isolated nucleic acid” as used herein also should be substantially separated from other naturally occurring nucleic acids that are on a different chromosome of the same organism. Specifically, an “isolated nucleic acid” means a composition in which the specified nucleic acid molecule is significantly enriched so as to constitute at least 10% of the total nucleic acids in the composition.

Some embodiments provide an isolated human gene or a portion thereof, or a product of either (e.g., mRNA, cDNA). As used herein, “gene” refers to the entire DNA sequence—including exons, introns, and non-coding transcription—control regions-necessary for production of a functional protein or RNA. A “portion” of a gene will generally be a nucleic acid whose nucleotide sequence comprises (1) a contiguous stretch of nucleotides that aligns perfectly with a region of the gene and that is unique within the human genome to that gene (e.g., at least 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 130, 140, 150, 160, 170, 180, 190, 200, 225, 250, 275, 300, 325, 350, 375, 400, 450, 500, 550, 600, 650, 700, 800, 900, 1000, 1200, 1400, 1600, 1800, 2000, 2500, 3500, 4000, 4500, 5000, 6000, 7000, 8000, 9000, 10000, 15000, 20000, 25000, 30000, 40000, 50000, 60000, 70000, 80000, 90000, 100000 or more contiguous nucleotides); and/or (2) a stretch of nucleotides that aligns with the gene at sufficient length and percent identity such that one skilled in the art would recognize the nucleic acid as coming from the gene or a variant of the gene rather than from an unrelated region of the genome (e.g., at least 20, 25, 30, 35, 40, 45, 50 or more nucleotides in length and at least 50%, 60%, 70%, 80%, 85%, 90%, 95%, or 99% identity). A “portion” of any other nucleic acid (e.g., mRNA, cDNA, oligonucleotide probe or primer, etc.) that can serve as a reference sequence is defined similarly (i.e., a nucleic acid whose nucleotide sequence comprises (1) a contiguous stretch of nucleotides that is unique within the human genome or transcriptome to that nucleic acid; and/or (2) a stretch of nucleotides of sufficient length and percent identity such that one skilled in the art would recognize the nucleic acid as coming from a variant of the nucleic acid rather than from an unrelated region of the genome or transcriptome).

In some embodiments the isolated gene (or portion or product thereof) of the invention comprises a variant listed in Table 1. A nucleic acid “comprising a variant” of the invention has its conventional meaning in the art. Those skilled in the art are familiar with various ways of determining whether a given nucleic acid “comprises a variant” of the invention. For example, in determining whether a sample contains an EGFR nucleic acid comprising the c.2525A>T variant listed in Table 1, one will generally: (1) determine whether the sample contains an EGFR nucleic acid (e.g., by sequencing and aligning with the canonical EGFR sequence, by amplifying EGFR nucleic acids using EGFR-specific primers, by hybridizing EGFR nucleic acids to a chip using EGFR-specific probes, etc.); and (2) determine what nucleotide residue is present in the detected nucleic acid at a position corresponding to the variant (e.g., by searching the sequence obtained in (1) for a region matching the EGFR sequence surrounding the variant (e.g., SEQ ID NOs 25 & 26) and determining the residue at the position of interest, by amplifying EGFR nucleic acids comprising the variant using primers comprising the variant, by identifying/quantifying EGFR nucleic acids comprising the variant using probes (e.g., TaqMan™ probes) comprising the variant, etc.).

Some embodiments provide isolated nucleic acids of specific lengths. Such nucleic acids may be at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 130, 140, 150, 160, 170, 180, 190, 200, 225, 250, 275, 300, 325, 350, 375, 400, 450, 500, 550, 600, 650, 700, 800, 900, 1000, 1200, 1400, 1600, 1800, 2000, 2500, 3000, 3500, 4000, 4500, 5000, 6000, 7000, 8000, 9000, 10000, 15000, 20000, 25000, 30000, 40000, 50000, 60000, 70000, 80000, 90000, 100000, 200000, 300000, 400000, 500000, 600000, 700000, 800000, 900000, 1000000 or more nucleotides in length or any range therein. Oligonucleotides (also called “oligos”) are relatively short nucleic acids and may be of any length listed above equal to or less than about 500. In some embodiments of the invention, oligos are between 5 and 500, 10 and 250, 18 and 150, 18 and 65, 22 and 250, 22 and 150, 22 and 65, 23 and 65, 25 and 65, and 30 and 65 nucleotides in length. In some embodiments the isolated nucleic acids (including oligo nucleotides) comprise a variant listed in Table 1.

Some embodiments provide isolated nucleic acids whose nucleotide sequences comprise at least 18, 19, 20, 21, 22, 23, 24, 25, 26, 27, 28, 29, 30, 31, 32, 33, 34, 35, 36, 37, 38, 39, 40, 45, 50, 60, 70, 80, 90, 100, 150, 200, 250, 300, 350, 400, 450, 500, 600, 700, 800, 900, 1000, 1200, 1400, 1600, 1800, 2000, 2500, 3000, 3500, or 3600 or more contiguous nucleotides of the sequence of SEQ ID NO:1, wherein the contiguous span comprises at least one variant listed in Table 1. Some embodiments provide isolated nucleic acids whose nucleotide sequences comprise at least 18, 19, 20, 21, 22, 23, 24, 25, 26, 27, 28, 29, 30, 31, 32, 33, 34, 35, 36, 37, 38, 39, 40, 45, 50, 60, 70, 80, 90, 100, 150, 200, 250, 300, 350, 400, 450, 500, 600, 700, 800, 900, 1000, 1200, 1400, 1600, 1800, 2000, 2500, 3000, 3500, 3600, 3700, or 3760 or more contiguous nucleotides of the sequence of SEQ ID NO:35, wherein the contiguous span comprises at least one variant listed in Table 1. Some embodiments provide isolated nucleic acids whose nucleotide sequences comprise at least 18, 19, 20, 21, 22, 23, 24, 25, 26, 27, 28, 29, 30, 31, 32, 33, 34, 35, 36, 37, 38, 39, 40, 45, 50, 60, 70, 80, 90, 100, 150, 200, 250, 300, 350, 400, 450, 500, 600, 700, 800, 900, 1000, 1200, 1400, 1600, 1800, 2000, 2500, 3000, 3500, 3600, 3700, 3800, or 3900 or more contiguous nucleotides of the sequence of SEQ ID NO:57, wherein the contiguous span comprises at least one variant listed in Table 1.

Some embodiments provide isolated nucleic acids whose nucleotide sequences comprise at least 18, 19, 20, 21, 22, 23, 24, 25, 26, 27, 28, 29, 30, 31, 32, 33, 34, 35, 36, 37, 38, 39, 40, 45, 50, 60, 70, 80, 90, 98, or 99 contiguous nucleotides of a sequence chosen from the group consisting of SEQ ID NOs 6, 10, 14, 18, 22, 26, 30, 34, 40, 44, 48, 52, 56, 62, 66, or 70, wherein the contiguous span comprises at least one variant listed in Table 1.

In some embodiments the isolated nucleic acid of the invention comprises a variant listed in Table 1 at a particular position along its length. In some of these embodiments the variant residue is at the center of said isolated nucleic acid, In other embodiments the variant residue is within 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100 or more nucleotide positions of the center of said isolated nucleic acid. In some embodiments the variant is no more than 5, no more than 4, no more than 3, no more than 2, or no more than 1 position from the center of the nucleic acid. As used herein, the “center” of a polynucleotide has the plain meaning given by those skilled in the art. The nucleotide (or pair of nucleotides) that, with respect to the linear sequence of nucleotides, has an equal number of residues on either side is the center of a polynucleotide. For instance, in the following oligonucleotide-5′-tcaaagtgctgggctccggtgcgttcggcacggtgtataagggactctggatcccagaa Agtgagaaagttaaaattcccgtcgctatcaaggaattaagagaagcaacatctccgaa a-3′ (SEQ ID NO:6)—the center of the oligo is the uppercase “A” residue because there are fifty-nine residues on each side. Sometimes a polynucleotide has an even number of residues and thus the “center” is the pair of nucleotides that has an equal number of residues on either side of the pair. Sometimes those skilled in the art will be interested in the center of a relevant region of a nucleic acid rather than the center of the entire nucleic acid. For instance, an oligonucleotide probe or primer might comprise only a portion that hybridizes to a target nucleic acid (with the rest of the probe or primer free, in a hairpin loop, etc.). In such a case, one may refer to the “center” of the hybridizing portion of the oligonucleotide as the residue (or pair of residues) that has an equal number of hybridizing nucleotides on each side. Conversely, one may refer to the center of, e.g., the hairpin as the residue (or pair of residues) that has an equal number of hairpin nucleotides on each side.

In some embodiments the variant listed in Table 1 is within 0, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100 or more nucleotide positions of the 5′ or 3′ end of a isolated nucleic acid of the invention. For example, a variant listed in Table 1 may appear at the extreme 5′ end of a nucleic acid of the invention. As another example, a variant listed in Table 1 may appear at the extreme 3′ end of a nucleic acid of the invention.

In some embodiments the invention provides an isolated nucleic acid (e.g., an oligonucleotide) of the invention that selectively hybridizes to or selectively amplifies a nucleic acid comprising a variant listed in Table 1. In some of these embodiments the isolated oligonucleotide hybridizes under stringent conditions to a nucleic acid whose nucleotide sequence comprises a variant listed in Table 1 but not to a nucleic acid whose nucleotide sequence comprises the wild-type residue. For example, the invention provides isolated an oligonucleotide that hybridizes under stringent conditions to a nucleic acid whose nucleotide sequence consists of the sequence of SEQ ID NO:4 but not to a nucleic acid whose nucleotide sequence consists of the sequence of SEQ ID NO:3. Those skilled in the art are familiar with various techniques for designing and using oligonucleotides with such specificity. In some embodiments this is accomplished by the oligo of the invention (1) encompassing a variant listed in Table 1 and (2) being of a such length and having the variant residue in such a position that the oligo will only hybridize under stringent (e.g., high stringency) conditions to nucleic acids that are highly homologous (sequence differences of 10%, 5%, 1% or less, including 0%).

The term “stringent conditions” is well-known in the art of nucleic acid hybridization and, as used herein, has its conventional meaning. The term. “high stringency hybridization conditions,” when used in connection with nucleic acid hybridization, means hybridization conducted overnight at 42 degrees C. in a solution containing 50% formamide, 5×SSC (750 mM NaCl, 75 mM sodium citrate), 50 mM sodium phosphate, pH 7.6, 5×Denhardt's solution, 10% dextran sulfate, and 20 microgram/ml denatured and sheared salmon sperm DNA, with hybridization filters washed in 0.1×SSC at about 65° C. The term “moderate stringency hybridization conditions,” when used in connection with nucleic acid hybridization, means hybridization conducted overnight at 37 degrees C. in a solution containing 50% formamide, 5×SSC (750 mM NaCl, 75 mM sodium citrate), 50 mM sodium phosphate, pH 7.6, 5×Denhardt's solution, 10% dextran sulfate, and 20 microgram/ml denatured and sheared salmon sperm DNA, with hybridization filters washed in 1×SSC at about 50° C. It is noted that many other hybridization methods, solutions and temperatures can be used to achieve comparable stringent hybridization conditions as will be apparent to skilled artisans.

In some embodiments the isolated nucleic acid (e.g., an oligonucleotide) selectively amplifies (together with another primer, under standard conditions and with standard reagents) a nucleic acid whose nucleotide sequence comprises a variant listed in Table 1, or a portion thereof comprising the variant, but not a nucleic acid whose nucleotide sequence comprises the wild-type residue, or a portion thereof comprising the wild-type residue. Often such a primer will, as above, only hybridize to target nucleic acids comprising the variant with at least some minimum level of sequence identity (e.g., 90%, 95%, 96%, 97%, 98%, 99%, or 100%). Those skilled in the art are familiar with other ways of designing primers to only amplify certain sequences, often with single nucleotide specificity. As a non-limiting example, one may design a primer such that a variant listed in Table 1 is at or near the 3′ end of the primer. Thus, under stringent conditions the primer might hybridize to both wild-type and variant of the target gene (e.g., EGFR) nucleic acids to some degree, while it's 3′ end will not hybridize (and thus not prime amplification) unless the target nucleic acid is an exact match.

The invention additionally provides an oligonucleotide probe set comprising 2 or more nucleic acid probes targeted to EGFR, HER2 and HER4 (and optionally PTEN). The probe set may comprise at least 2, 3, 4, 5, 6, 7, 8, 9, 10, 15, 20, 25, 30, 35, 40, 45, 50, 60, 70, 80, 90, 100, 150, 200, 250, 300, 350, 400, 450, 500, 600, 700, 800, 900, 1000, 1500, 2000, 2500, 3000, 3500, 4000, 4500, 5000, 6000, 7000, 8000, 9000, 10000, 15000, or 20000 or more probes targeted to EGFR, HER2 and HER4 (and optionally PTEN). The invention also provides a microarray comprising such a probe set.

On aspect of the invention provides an isolated human protein or peptide, or a portion thereof, comprising a variant listed in Table 1. The term “isolated polypeptide” as used herein is defined as a polypeptide molecule that is present in a form other than that found in nature. Thus, an isolated polypeptide can be a non-naturally occurring polypeptide. For example, an “isolated polypeptide” can be a “hybrid polypeptide.” An “isolated polypeptide” can also be a polypeptide derived from a naturally occurring polypeptide by additions or deletions or substitutions of amino acids. An isolated polypeptide can also be a “purified polypeptide” which is used herein to mean a composition or preparation in which the specified polypeptide molecule is significantly enriched so as to constitute at least 10% of the total protein content in the composition. A “purified polypeptide” can be obtained from natural or recombinant host cells by standard purification techniques, or by chemically synthesis, as will be apparent to skilled artisans. “Isolated polypeptide” also includes antibodies, including monoclonal, polyclonal, humanized, and fully human antibodies.

A “portion” of a protein will generally be a polypeptide whose amino acid sequence comprises (1) a contiguous stretch of amino acids that is unique to that protein within the human proteome (e.g., at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 130, 140, 150, 160, 170, 180, 190, 200, 225, 250, 275, 300, 325, 350, 375, 400, 450, 500, 550, 600, 650, 700, 800, 900, 1000, 1200, 1400, 1600, 1800, 2000, 2500, 3500, 4000, 4500, 5000, 6000, 7000, 8000, 9000, 10000, 15000, 20000, 25000, 30000, 40000, 50000, 60000, 70000, 80000, 90000, 100000 or more contiguous amino acids); and/or (2) a stretch of amino acids of sufficient length and percent identity such that one skilled in the art would recognize the polypeptide as coming from a variant of the protein rather than from an unrelated protein (e.g., at least 20, 25, 30, 35, 40, 45, 50 or more amino acids in length and at least 50%, 60%, 70%, 80%, 85%, 90%, 95%, or 99% identity).

Some embodiments provide isolated polypeptides of various lengths comprising at least one variant of the invention. Such polypeptides may be at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 130, 140, 150, 160, 170, 180, 190, 200, 225, 250, 275, 300, 325, 350, 375, 400, 450, 500, 550, 600, 650, 700, 800, 900, 1000, 1025 or more amino acids in length or any range therein. In some embodiments the polypeptide is any length listed above equal to or less than about 500. In other embodiments polypeptides are between 5 and 500, 8 and 250, 18 and 150, 18 and 65, 22 and 250, 22 and 150, 22 and 65, 23 and 65, 8 and 65, 10 and 50, or 10 and 35 amino acids in length.

Some embodiments provide isolated polypeptides whose amino acid sequences comprise at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 105, 110, 115, 120, 130, 140, 150, 160, 170, 180, 190, 200, 225, 250, 275, 300, 325, 350, 375, 400, 450, 500, 550, 600, 650, 700, 800, 900, 1000, or 1025 contiguous amino acids of the sequence of SEQ ID NO:2, wherein the contiguous span comprises at least one variant listed in Table 1. Some embodiments provide isolated polypeptides whose amino acid sequences comprise at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, or 51 contiguous amino acids of the sequence of SEQ ID NOs 48-61, wherein the contiguous span comprises at least one variant listed in Table 1. Still other embodiments provide isolated nucleic acids whose nucleotide sequences comprise at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, or 49 contiguous amino acids of the sequence of SEQ ID NOs 63 & 84-97, wherein the contiguous span comprises at least one variant listed in Table 1.

Another aspect of the invention provides antibodies that bind polypeptides encoded by EGFR, HER2, HER4 or PTEN. In some embodiments the antibodies bind specifically to a polypeptide variant of the invention and do not bind specifically to the corresponding wild-type protein. Such antibodies may be monoclonal, polyclonal, murine, humanized murine, fully humanized, antibody fragments, etc. Such antibodies may be generated based on the present novel sequence disclosures in Table 1 and FIG. 6 combined with various routine techniques known to those skilled in the art. For example, antibodies binding specifically to the variants of the invention and not to the wild-type protein may be produced using peptides comprising an amino acid variant listed in Table 1 as immunogens (generally conjugated to some carrier such as KLH). The invention also provides hybridoma cell lines secreting antibodies of the invention.

Another aspect of the invention provides kits comprising reagents suitable for detecting, measuring, sequencing, or otherwise analyzing EGFR, HER2, HER4, and optionally PTEN. In some embodiments, the kit includes (a) an EGFR reagent for evaluating the status of EGFR (e.g., primers and/or probes for evaluating expression or sequence) in a sample; (b) a HER2 reagent for evaluating the status of HER2 (e.g., primers and/or probes for evaluating expression or sequence) in a sample; (c) a HER4 reagent for evaluating the status of HER4 (e.g., primers and/or probes for evaluating expression or sequence) in a sample; and optionally (d) a PTEN reagent for evaluating the status of PTEN (e.g., primers, probes and/or antibody for evaluating expression or sequence) in a sample. It is contemplated that the reagents for evaluating the level of expression or activity of any of EGFR, HER2, HER4, or PTEN can be one or more nucleic acids. The nucleic acids may be complementary to all or part of the gene or its product and they can be used in hybridization reactions, such as for amplification (primers), primer extensions, nuclease protection assays, Northern blotting, or with an array or other structure. Alternatively, antibodies against EGFR, HER2, HER4, or PTEN can be used, for example, in Western blotting, ELISAs, other sandwich assays, antibody arrays, IHC, or FACS analysis. The antibody may be a monoclonal or a polyclonal antibody. It is contemplated that kits of the invention may comprise 1, 2, 3, 4, 5, 6 or more HER2 reagents and 1, 2, 3, 4, 5, 6 or more PTEN reagents. The kits of the invention may further comprise additional reagents suitable for performing hybridization and/or amplification reactions or for performing antibody analysis.

The kit may include a carrier for the various components of the kit. The carrier can be a container or support, in the form of, e.g., bag, box, tube, rack, and is optionally compartmentalized. The carrier may define an enclosed confinement for safety purposes during shipment and storage. The kit may also include instructions on the interpretation of the results of the test performed—e.g., instructions explaining that activated status for any of EGFR, HER2 or HER4 or optionally low or negative status for PTEN indicates low or decreased likelihood of response to anti-HER2 receptor therapy (and optionally indicates normal or increased likelihood of response to KI therapy).

While the present invention is discussed with respect to the treatment of cancer, it is contemplated that the present invention has applications generally to any disease or condition involving HER2 activity, particularly any diseases or conditions characterized by a relatively high activity or expression level of HER2. Furthermore, any method used or discussed herein with respect to the detection of HER2 overexpression in cancer cells may be implemented with respect to the detection of PTEN expression, and vice versa.

### Example 1

It has been discovered that either loss of PTEN expression or a somatic mutation in the kinase domain of an EGFR-family member predicts non-response to trastuzumab in patients with metastatic breast cancer. Specifically, a kinase domain mutation in EGFR, HER2, or HER4 or loss of PTEN expression by IHC confers resistance to trastuzumab response. The data are as follows:

The kinase domains of EGFR, HER2 and HER4 were fully sequenced in tumor 108 samples obtained from metastatic breast cancer patients (with accompanying objective response data for each patient). Kinase mutations were found in EGFR (5/67), HER2 (3/76), and HER4 (2/71), as detailed in Table 1 above. There were no drug responders among mutation carriers. This was a significantly lower response rate than that seen in non-mutants (0/10 vs. 13/37, p-value=0.0166).

Tumor samples were further evaluated for PTEN expression using IHC (anti-PTEN antibody from Cell Signaling Technology). Loss of PTEN expression was found to predict trastuzumab resistance: 1/23 responders in the PTEN negative group vs. 22/82 in the PTEN positive group (p-value 0.02). PTEN scoring was dichotomous, with a score of <10 meaning “PTEN negative” status.

These data were then combined in a set of 105 patients. In the combined analysis patients with either loss of PTEN expression or mutations in one of the EGFR-family members (“pathway mutants”) were less likely to respond to trastuzumab: 1/30 responders in the pathway mutant group vs. 22/75 in the group with wild-type pathway activity (p-value 0.002). Response for all aspects of the study was defined by RECIST criteria.

It is specifically contemplated that any embodiment of any method or composition of the invention may be used with respect to any other method or composition of the invention.

In the context of genes and gene products, the name of the gene is generally italicized herein following convention. In such cases, the italicized gene name is generally to be understood to refer to the gene (i.e., genomic), its mRNA (or cDNA) product, and/or its protein product. Generally, though not always, a non-italicized gene name refers to the gene's protein product.

The use of the term “or” in the claims is used to mean “and/or” unless explicitly indicated to refer to alternatives only or the alternative are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

Unless otherwise defined, all technical and scientific terms used herein have the same meaning as commonly understood by one of ordinary skill in the art to which this invention pertains. In case of conflict, the present specification, including definitions, will control. In addition, the materials, methods, and examples are illustrative only and not intended to be limiting.

Other features and advantages of the invention will be apparent from the preceding detailed description and from the following claims.

1-14. (canceled)

15. A method of optimizing treatment of a cancer patient comprising evaluating EGFR, HER2 and HER4 status in a sample from said patient and either (a) recommending a treatment regimen that does not comprise an anti-HER2 receptor agent if an activated status is detected in any of EGFR, HER2 or HER4 in said sample or (b) recommending a treatment regimen comprising an anti-HER2 receptor or kinase inhibitor agent if an activated status is not detected in all of EGFR, HER2 and HER4 in said sample.

16. The method of claim 15, further comprising evaluating PTEN status in a sample from the patient and (a) recommending a treatment regimen that does not comprise an anti-HER2 receptor agent if an activated status is detected in any of EGFR, HER2 or HER4 in said sample or a low or negative status is detected in PTEN in said sample or (b) recommending a treatment regimen comprising an anti-HER2 receptor agent if an activated status is not detected in all of EGFR, HER2 and HER4 in said sample and a low or negative status is not detected in PTEN in said sample.

17. The method of claim 16, further comprising evaluating HER2 amplification/overexpression and either (a) recommending a treatment regimen that does not comprise an anti-HER2 receptor agent if HER2 overexpression is not detected in said sample, shows an activated status for each of EGFR, HER2 and HER4 is detected in said sample, or a low or negative status for PTEN is detected in said sample, or (b) recommending a treatment regimen that includes an anti-HER2 receptor agent if HER2 overexpression is detected in said sample, an activated status for each of EGFR, HER2 and HER4 is not detected in said sample, and a low or negative status for PTEN is not detected in said sample.

18-19. (canceled)

20. An isolated nucleic acid comprising at least 18 consecutive nucleotides of any one of SEQ ID NO:1, SEQ ID NO:35, or SEQ ID NO:57, wherein said at least 18 consecutive nucleotides comprise at least one of the nucleotide variants listed in Table 1.

21-25. (canceled)

26. A kit comprising at least one oligonucleotide probe, wherein each probe specifically hybridizes under stringent conditions to EGFR, HER2 or HER4 comprising at least one variant listed in Table 1, but not to EGFR, HER2 or HER4 lacking such variant.

27-30. (canceled)

31. The method of claim 15, wherein one of said genes has an activated status if said gene harbors an activating mutation.

32. The method of claim 31, wherein said activating mutation is found in the kinase domain of said gene.

33. The method of claim 32, wherein said activating mutation is found at a locus listed in Table 1.

34. The method of claim 15, further comprising evaluating HER2 amplification/overexpression and either (a) recommending a treatment regimen that does not comprise an anti-HER2 receptor agent if HER2 overexpression is not detected in said sample or an activated status for each of EGFR, HER2 and HER4 is detected in said sample, or (b) recommending a treatment regimen that includes an anti-HER2 receptor agent if HER2 overexpression is detected in said sample and an activated status for each of EGFR, HER2 and HER4 is not detected in said sample.

35. A method of optimizing treatment of a cancer patient comprising evaluating HER4 status in a sample from said patient and either (a) recommending a treatment regimen that does not comprise kinase inhibitor therapy if an activating mutation in HER4 is detected in said sample or (b) recommending a treatment regimen comprising kinase inhibitor therapy if an activating mutation in HER4 is not detected in said sample.
